(19) United States
(12) Patent Application Publication
(10) Pub. No.: US 2022/0049310 A1
(43) Pub. Date: Feb. 17, 2022

(54) Use of ZNF124 Gene in Early Screening or Auxiliary Diagnosis of Retinitis Pigmentosa Disease

(71) Applicant: Sichuan Provincial People's Hospital, Sichuan (CN)

(72) Inventors: Xianjun Zhu, Sichuan (CN); Zhenglin Yang, Sichuan (CN); Yeming Yang, Sichuan (CN); Wanli Tian, Sichuan (CN); Wenjing Liu, Sichuan (CN)

(21) Appl. No.: 17/416,869

(22) PCT Filed: Oct. 20, 2020
(86) PCT No.: PCT/CN2020/122230

§ 371 Date: Jun. 21, 2021

(30) Foreign Application Priority Data
Feb. 25, 2020 (CN) 2020101148904
Feb. 25, 2020 (CN) 2020101149038

(51) Int. Cl. C12Q 1/6883

(52) U.S. Cl. CPC C12Q 1/6883; C12Q 2600/156

(57) ABSTRACT

The present disclosure discloses a use of a ZNF124 gene in early screening or auxiliary diagnosis of retinitis pigmentosa disease. The present disclosure also discloses a use of a detection reagent for detecting ZNF124 gene mutation in preparation of a reagent or a kit for early screening or auxiliary diagnosis of the retinitis pigmentosa disease. Research of the present disclosure finds that the mutation of the ZNF124 gene is related to the retinitis pigmentosa disease, early screening or auxiliary diagnosis can be performed on the retinitis pigmentosa disease by detecting the mutation of the ZNF124 gene.

### CROSS-REFERENCE TO RELATED APPLICATIONS

This disclosure claims priority to Chinese Patent Application No. 2020101148904 filed with the Chinese Patent Office on Feb. 25, 2020, entitled “Use of ZNF124 Gene in Early Screening or Auxiliary Diagnosis of Retinitis Pigmentosa Disease”, and claims priority to Chinese Patent Application No. 2020101149038 filed with the Chinese Patent Office on Feb. 25, 2020, entitled “Method and Use for Constructing a Retinitis Pigmentosa Disease Model by Utilizing a Gm20541 Gene”, which are incorporated herein by reference in their entirety.

### TECHNICAL FIELD

The present disclosure involves the technical field of gene detection, in particular, to the use of a ZNF124 gene in early screening or auxiliary diagnosis of retinitis pigmentosa diseases.

### BACKGROUND ART

Retinitis pigmentosa (RP) is a genetic retinitis disease caused by abnormal photoreceptor cells (comprising rod cells and cone cells). The incidence of RP worldwide is about 1/5000-1/3500. In our country, the incidence of RP is increasing year by year, about 1/1000, which is one of the important causes of blindness. A typical RP patient first has night blindness and narrow visual field due to function defect of rod cells, and gradually develops a tubular visual field until blindness; retinal pigmentation can be seen from fundus examination. The pathogenesis of RP is extremely complex, and involves many different biological metabolic pathways. The protein encoded by its pathogenic genes is mainly involved in the processes of light transmission, maintenance of the structure of photoreceptor cell, mRNA splicing and the like. Mutations in the encoding genes of various proteins in different biological pathways make the function of the proteins impaired, which may lead to the abnormality of photoreceptor cells and thus cause RP. In terms of pathology, typical RP mainly affects rod cells, and causes death of rod cells and then secondary death of cone cells, and is mainly expressed as photoreceptor damage and degeneration, the gradual thinning and even the disappearance of the outer nuclear layer of the retina, and corresponding pathological changes in the outer network layer of retina and other related cell layers.

RP has obvious genetic predisposition. According to genetic methods, RP can be mainly divided into autosomal dominant RP (adRP: 15-25%), autosomal recessive RP (arRP: 5-25%) and X-linked RP (XLRP: 10-15%). In addition, RP also has some rare atypical Mendelian inheritance modes, such as two-gene inheritance, mitochondrial inheritance, and Y-linked inheritance. At present, it is reported that there are 70 gene mutations which are related to RP, but this can only explain about 50% of the cases, and there are still some pathogenic genes that have not been found. Therefore, it is critical for early screening, prevention and control of RP to further search for and expand RP-related pathogenic genes and their mutations.

### SUMMARY

The present disclosure provides a use of a detection reagent for detecting ZNF124 gene mutation in preparation of a reagent or a kit for early screening or auxiliary diagnosis of retinitis pigmentosa diseases.

The present disclosure provides a detection reagent for detecting ZNF124 gene mutation for use in early screening or auxiliary diagnosis of retinitis pigmentosa diseases.

In one or multiple embodiments, the mutation of the ZNF124 gene detected by the detection reagent comprises: c.219-1G>-.

In one or multiple embodiments, the ZNF124 gene mutation c.219-1G>- is a homozygous mutation.

In one or multiple embodiments, the detection reagent is selected to be applicable to any one of or a combination of the following methods for detecting ZNF124 gene mutation: a restriction fragment length polymorphism method, denaturing gradient gel electrophoresis, allele-specific PCR, a DNA sequencing method, a DNA chip detection method, a time-of-flight mass spectrometry and a single-strand conformation polymorphism analysis.

In one or multiple embodiments, the DNA sequencing method is a whole exon sequencing method or a Sanger sequencing method.

The present disclosure provides a method for early screening or auxiliary diagnosis of the retinitis pigmentosa diseases of a subject, comprising:

detecting the mutations of the ZNF124 gene in the subject's sample.

In one or multiple embodiments, the mutations of the ZNF124 gene comprise c.219-1G>-.

In one or multiple embodiments, the mutations of the ZNF124 gene comprise the homozygous mutation of c.219-1G>-.

In one or multiple embodiments, the detection of the mutations of the ZNF124 gene in the sample of the subject is carried out by using a detection reagent.

In one or multiple embodiments, the sample is the body fluid, tissue or hair of the subject.

In one or multiple embodiments, the subject is a human.

In one or multiple embodiments, the body fluid is selected from blood, saliva or semen.

The present disclosure provides a reagent or a kit for early screening of retinitis pigmentosa diseases, which contains the detection reagent for detecting ZNF124 gene mutation.

In one or multiple embodiments, the mutations of the ZNF124 gene detected by the detection reagent comprise c.219-1G>-.

In one or multiple embodiments, the detection reagent is selected to be applicable to any one of or a combination of the following methods for detecting ZNF124 gene mutation: a restriction fragment length polymorphism method, denaturing gradient gel electrophoresis, allele-specific PCR, a DNA sequencing method, a DNA chip detection method, a time-of-flight mass spectrometry and a single-strand conformation polymorphism analysis.

In one or multiple embodiments, the DNA sequencing method is a whole exon sequencing analysis or a Sanger sequencing method.

The present disclosure provides a reagent or a kit for auxiliary diagnosis of retinitis pigmentosa diseases, which contains the detection reagent for detecting ZNF124 gene mutation.

In one or multiple embodiments, the mutations of the ZNF124 gene detected by the detection reagent comprise c.219-1G>-.

In one or multiple embodiments, the detection reagent is selected to be applicable to any one of or a combination of the following methods for detecting ZNF124 gene mutation: a restriction fragment length polymorphism method, a denaturing gradient gel electrophoresis, allele-specific PCR, a DNA sequencing method, a DNA chip detection method, a time-of-flight mass spectrometry and a single-strand conformation polymorphism analysis.

In one or multiple embodiments, the DNA sequencing method is a whole exon sequencing analysis or a Sanger sequencing method.

The present disclosure provides a reagent or a kit for early screening or auxiliary diagnosis of retinitis pigmentosa diseases, which contains the detection reagent for detecting ZNF124 gene mutation.

In one or multiple embodiments, the mutations of the ZNF124 gene detected by the detection reagent comprise c.219-1G>-.

In one or multiple embodiments, the detection reagent is selected to be applicable to any one of or a combination of the following methods for detecting ZNF124 gene mutation: a restriction fragment length polymorphism method, a denaturing gradient gel electrophoresis, allele-specific PCR, a DNA sequencing method, a DNA chip detection method, a time-of-flight mass spectrometry and a single-strand conformation polymorphism analysis.

In one or multiple embodiments, the DNA sequencing method is a whole exon sequencing analysis or a Sanger sequencing method.

In one or multiple embodiments, the type of the sample detected by the reagent or kit is the body fluid, tissue or hair from the subject to be tested.

In one or multiple embodiments, the subject to be tested is a human.

In one or multiple embodiments, the body fluid is selected from blood, saliva or semen.

In one or multiple embodiments, the ZNF124 gene mutation is a mutation on exon 4 of the ZNF124 gene, and preferably the mutation on exon 4 is a homozygous mutation.

In one or multiple embodiments, the retinitis pigmentosa diseases comprise night blindness and/or impaired visual field.

### BRIEF DESCRIPTION OF DRAWINGS

In order to more clearly illustrate technical solutions of embodiments of the present disclosure, accompanying drawings which need to be used in the embodiments will be introduced briefly below, and it should be understood that the accompanying drawings below merely show some embodiments of the present disclosure, therefore, they should not be considered as limitation to the scope, and those ordinarily skilled in the art still could obtain other relevant drawings according to these accompanying drawings, without using any inventive effort.

FIG. 1 shows the results of ophthalmological examinations for a proband in the RP074 family and his/her parents; in the figure, A, fundus examination reveals that the patient's retina has pigmentation; B, macular OCT examination reveals that the patient's retinal neuroepithelial layer is thinning, the reflex of the connection layer of the inner and outer segments of the photoreceptor disappears, and the pigment epithelium layer atrophies.

FIG. 2 shows the visual field and ERG test results of the proband in the RP074 family; in the figure, A-B, the visual field test shows that the peripheral visual field is missing, and shows a tubular visual field; C, the ERG test reveals that the wave peaks of ERG a- and b-waves both greatly lower in the dark light, and the function of the patient's photoreceptor cell is impaired.

FIG. 3 shows the family tree of RP074 and the mutation site of ZNF124 gene; in the figure, A, IV:4 and IV:5 are the proband, IV:4, IV:5 and his/her parents III:3, III:4 were all subjected to WES analysis; B, the results of exon sequencing analysis reveal that his/her parents III:3 and III:4 all carry ZNF124 c.219-1G>-heterozygous deletion mutation; C, Sanger sequencing verifies that IV:4 and IV:5 carry homozygous mutations, and his/her parents III:3 and III:4 all carry heterozygous deletion mutation, IV:6 is not diseased and does not have such deletion mutation.

FIG. 4 shows the family tree of RP131 and the mutation site of ZNF124 gene; in the figure, A shows RP131 family tree and the genotypes of family members, and III:3 is the proband; B, Sanger sequencing analysis shows that the proband III:3 carries the ZNF124 c.219-1G>-homozygous deletion mutation; his/her parents 11:3, 11:4 and sister III:4 are heterozygous carriers.

FIG. 5 shows the cDNA sequencing results of the proband of RP074 family and normal controls, the cDNA of the ZNF124 of the proband IV:4 misses 10 bases (red).

FIG. 6 is a schematic diagram showing the mutation of ZNF124 gene; A, the deletion mutation site of the ZNF124 gene is located at the splicing site of its exon 4; B, this mutation leads to mRNA splicing errors, which renders fragment deletions and base frameshifts; C, this mutation ultimately leads to ZNF124 protein translation errors.

FIG. 7 shows the study on ZNF124 gene expression; A-B, RT-PCR experiments in various mouse tissues show that the homologous gene Gm20541 of ZNF124 in mice is widely expressed in brain tissue, retina, liver, heart, muscle and other tissues; C, ZNF124 antibody can specifically recognize the ZNF124 protein transfected and expressed in 293T cells, which is the same size as the fragment recognized by the tag protein Flag antibody; D, the ZNF124 expression plasmid is transfected in 293T cells, and the ZNF124 and its tag Flag are respectively stained by immunohistochemical staining, and the results show that the ZNF124 protein is located in the nucleus; E, the retina section of rhesus monkey is stained with ZNF124 antibody, and ZNF124 protein is mainly located in the nucleus of optic cells, bipolar cells and ganglion cells.

FIG. 8 shows the result of identification of the first filial generation mouse by middle- and long-distance FOR; and

in the figure, A: the long arm of the end 5′ is amplified using the primer pair Gm 5′LRF and SA3′R, and the amplification product is 2.3 kb, wherein A2, 3, 6, 9, 10 and B1 are positive; B: the long arm of the end 3′ is amplified using the primer pair NeoF and Gm 3′LRR, the amplification product is 2.6 kb, wherein A2, 3, 6, 9, 10, B6, 9, ES1G, ES2G are positive heterozygotes, and +/+ are wild-type controls.

FIG. 9 shows the identification of Gm20541 gene knockout mice; and

A: the genotype identification results of Gm20541 gene knockout mice; B: real-time quantitative PCR analysis of gene knockout efficiency in the retina of Gm20541 gene knockout mice proves that Gm20541 is no longer expressed in the retina of the knockout mice; SixKO or cKO refers to Gm20541 gene knockout homozygous mice; Ctr refers to a wild type; Het refers to heterozygotes.

FIG. 10 shows the result of a scotopic electroretinogram (ERG) detection; and

in the figure, A-C: the trajectory graph of the scotopic electroretinogram of Gm20541 gene knockout mice under different light intensities; D: the a-wave statistics of the scotopic electroretinogram of Gm20541 gene knockout mice under different light intensities; E: the b-wave statistics of the scotopic electroretinogram of Gm20541 gene knockout mice under different light intensities; Scotopic amplitude: peak value in dark light measurement; Flash intensity; a-wave; and b-wave.

FIG. 11 shows the result of a light-adapted electroretinogram (ERG) detection; and

in the figure, A-B: the trajectory graph of the light-adapted electroretinogram of Gm20541 gene knockout mice under different light intensities; C: the trajectory graph of the light-adapted flicker electroretinogram of Gm20541 gene knockout mice under the light intensity of 20 cd·s/m²; D: the statistics of a-wave of light-adapted electroretinogram of Gm20541 gene knockout mice under different light intensities; E: the statistics of b-wave of light-adapted electroretinogram of Gm20541 gene knockout mice under different light intensities; F: light-adapted flicker electroretinogram statistics of Gm20541 gene knockout mice under the light intensity of 20 cd·s/m²; SixKO or cKO refers to Gm20541 gene knockout homozygous mice; Ctr refers to a wild-type; and Het refers to heterozygous.

FIG. 12 shows the results of immunohistochemical staining for retinal sections of mice which Gm20541 gene is specifically knocked out from retinal precursor cells; mouse age: 4 months; OS: outer-segment; IS: Inner-segment; ONL: Outer nuclear layer; and INL: Inner nuclear layer; and

in the figure, A: the H&E staining results of the paraffin section of the retina in mice which Gm20541 gene is specifically knocked out from retinal precursor cells, wherein the outer nuclear layer and inner nuclear layer are both thinned; B: statistics on the thickness of the outer nuclear layer of the retina of Gm20541 knockout mice in different parts.

FIG. 13 shows the results of IHC staining of mice which Gm20541 gene is specifically knocked out from retinal precursor cells, which indicate that the outer segment of the retina of the Gm20541 knockout mice become shorter and degenerate. SixKO refers to Gm20541 gene knockout homozygous mouse; Ctr refers to a wild-type mouse; DAPI (4′,6-diamidino-2-phenylindole): nuclear dye 4′,6-diamidino-2-phenylindole; Rhodopsin: rhodopsin antibody; Merge: the two colors overlapping each other.

### DETAILED DESCRIPTION OF EMBODIMENTS

In order to make objects, technical solutions and advantages of the embodiments of the present disclosure clearer, the technical solutions in the embodiments of the present disclosure will be described clearly and completely below. If specific conditions are not indicated in the examples, it shall be carried out in accordance with conventional conditions or the conditions recommended by manufacturers. If the manufacturers of the reagents or instruments used are not specified, they are all conventional products that can be purchased in the market.

The present disclosure provides a use of a ZNF124 gene in early screening or auxiliary diagnosis of retinitis pigmentosa diseases. Research of the present disclosure reveals that the mutation of the ZNF124 gene is related to the retinitis pigmentosa diseases, early screening or auxiliary diagnosis of retinitis pigmentosa diseases can be performed by detecting the mutation of the ZNF124 gene, and a new solution is provided for early screening or diagnosis of retinitis pigmentosa diseases.

The embodiment of the present disclosure provides the use of a detection reagent for detecting ZNF124 gene mutations in preparation of a reagent or a kit for early screening or auxiliary diagnosis of retinitis pigmentosa diseases.

The ZNF124 gene is a new gene encoding zinc finger protein. Zinc finger proteins are a type of transcription factor with finger-like domains, which play an important role in gene regulation. The ZNF124 protein can pass through the nuclear pore and enter the nucleus and act as a transcription factor regulating the expression of other genes. Research of the present disclosure reveals that the ZNF124 gene mutation can lead to the retinitis pigmentosa diseases, and the ZNF124 gene mutation is related to the retinitis pigmentosa diseases, early screening or auxiliary diagnosis of retinitis pigmentosa diseases can be achieved by detecting the mutation of ZNF124 gene. Therefore, the present disclosure provides a new use for the detection reagents for detecting the ZNF124 gene mutation, and the type of detection reagents can be used in preparation of a reagent or a kit for early screening or auxiliary diagnosis of retinitis pigmentosa diseases, which provides a new detecting method for early screening or diagnosis of retinitis pigmentosa diseases.

In an optional embodiment, the mutation of the ZNF124 gene detected by the detection reagent comprises: c.219-1G>-.

The present disclosure has performed exon sequencing on two members of a RP family, and it is found that exon 4 of the ZNF124 gene (NM_003431) of the member of the RP family with a retinitis pigmentosa disease has a homozygous deletion mutation c.219-1G>-, that is, compared with normal individuals, for the member with a retinitis pigmentosa disease, a G at the first site upstream of the 219th base in the encoding region of the gene is deleted, and the 219th base is exactly in the first site of the exon 4. The specific mutation sites are shown as follows, the underlined portion is the exon sequence, and the first italicized letter at the fourth underlined portion is the mutation site:

| SEQ ID NO. 1 | |
|---|---|
| 1 | tgataggtgc gtgggcccag actccccttt tctctgtgat tggagtggat gccaagagtg |
| 61 | aggagaagcc agacagtggg agcgagcact tccaagcctg tgggggtggg ggaaccttcc |
| 121 | tgagccctcc agagtgcaga gatgcctggg tctgcagcca tggcggctgt gcccaggagg |
| 181 | atgggcctcc tgcctgctcc agctcccaag agcacaggga tgcccagctc acctatgggt |
| 241 | caacccccaa gaacatttat aatttctcta gaatggcata acccttccaa aagctgacgg |
| 301 | ggaccttggg aatcttcttc cagagggtcc tgggtcaggg atattggcct ggctgtgaga |
| 361 | cagacaacct cctaccctcc ctgtgcctcc tgggccaaga cgtggaagcc tcatcaggat |
| 421 | gcactgccac tggcaacagg ccaagaacgg ggtctgcttg gggggcgcag gtggttcggg |
| 481 | ggtcccagga gaattgagaa cttcctgcag acaagatgcc tctttttcct catttttggt |
| 541 | ccaactgata cactggatag tgagataaac cttacatttt aatttttatg tattttttca |
| 601 | ttatatgcaa aatgtatact taaacagaca gtggatttga agggaaaagt atttgagatt |
| 661 | cattctttca agtgtcattt agtcctatat acataggttt atatatatta tatatatagt |
| 721 | gtatatatat agcatatata gtaatgtata tatgtagtgt atagtatata tgtagtgtat |
| 781 | atatactata tatgtgtata tatagtatat atgtatatat gcatacatat gtatatagta |
| 841 | tacatgcata catatgtata tagtatagat acatatataa atatatatgc tatatgctat |
| 901 | atataggact atatatatat acacatacac acacgcgtat atatatagtc ctatatacat |
| 961 | agtcctatat acagatttgc cctatgtgta acaactacgt agataaaaaa gttatgaaat |
| 1021 | tgtttttggt tttacaatgc tatattaaaa acattgaaat tcaccaattg aacaatctat |
| 1081 | gcaagggtat tatggttttt tgttattgtt gtttaagtcc tgaaatacag agataagagc |
| 1141 | tgagtgagca gccactggtg gagaaacagt cttcacgggg ccagtatttt tctgccccgt |
| 1201 | ctcatttggc tgcggatcaa cacatgtcat ttagtacaaa caagcaaaca aaaatagcaa |
| 1261 | tatgcttgtg tgcctacccc agatgcttta tgtgcaataa tgaaatgagg aaggggaaga |
| 1321 | taagagaatg gagaaaagga tactgtacca gccagaatgt ggagtcagaa tgtggtgtca |
| 1381 | gaatgtggag tcagaatgtg atgaaatcac attgtgggtt tctacatgag aatgtctgct |
| 1441 | ggagttgtag gaagaggtcc tgggtaaagg agcaggtgtg ttcttagaag gccctcctgt |
| 1501 | ctgctgctgg aacacatcac ctgtttattc gtcctccatt tcccgctttg caggtgtgtc |
| 1561 | agtttcactg agtgaccgtc cgtaaaatcc aaatctgatt tgcttcatgg ccaaaccctg |
| 1621 | ccattcacca taggcttcca ctccttcatt cagtgatgtg tacttcttcc ccttcaactg |
| 1681 | cgccccagaa ccatcctacc ctccaccttc aaattcacag aattgttgtt ctgtcttgac |
| 1741 | tcaaactttt ttcctgccac agcagagcac tgaaagtctc ttaagttaca gcttcacaac |
| 1801 | agaagtctaa agtctgcgca gaactagcac atctgcagcc gtaggagcag cagaatgacc |
| 1861 | aggcagcaga gctttattcc atgttttcat ctccattcct acaacagcag cggcagcagc |
| 1921 | aacaactgct actacttcta attgtggtaa aaatacttag aataataatg cataatgaca |
| 1981 | attgtagttg acatttttgg tttatatatg attatttgta atgttctaca atttaccttt |
| 2041 | agtaatatac acataacttt tgcataaaca tcaggaccct aaaacaccac acctgatgat |
| 2101 | tgaatctcac ctatggacct aaggtcttca catgctggtg caggccctct caccactcca |
| 2161 | tctccaaaca gaggatatgg tccaccttct gagtcaatat agtagccctg acactcacat |
| 2221 | gatctatttg aggtcttgga aaacaagctt gccttgttaa aaatcaaagg atgggccggg |
| 2281 | cacggtggct cacgcctgta atcccagcac tttgggaggc cgaggcgggc tgatcacgag |
| 2341 | gtcaggagat ggagaccatc ctgactaaca cgatgaaacc ccatctctac taaaaataca |
| 2401 | aaaaaattag ccgggcatgg tggccggcgc ctgtagtccc agctactcgg gaggctgagg |
| 2461 | caggagaatg gcgtgaaccc gggaggcgga gcttgcagtg agccgagatc acaccactgc |
| 2521 | actccagcct ggatgacaca gcgagactcc atcttaaaaa aaaaaaaaaa aaaaaaaatc |
| 2581 | aaagaatggc ttcaaattgt agtcagctgt ttcgttaaat tgcagcagga agaaattttt |
| 2641 | ctcagcaggt ctattttcat gtcacttgct ttcagagtaa gaggttattt cagacacagt |
| 2701 | ggtttgattt agtctttgtt gaaaatcata aatagcctac aaacaacaaa aagggatgaa |
| 2761 | taggatttgc gttcatgtgg agtgcgtcat ctgtgctttt aaatttatcc tatgaaaaga |
| 2821 | aataaaaaat atgtaatatc gaacattggt ttcctaaatt agcaaatcga ccatttcctc |
| 2881 | aagttattcc acgttcttcc aagatgtgtc agaagcattc tctggaggca gttgttccaa |
| 2941 | atggtgaaga gtactagatg gatcccaatc tcacaggatt cttgcttcta ttgcagaaga |
| 3001 | gttactttcc aggacttcaa aggtttagag aagctgcttg attcgtgtgg cttgcagagg |
| 3061 | tgcatgggct ttggagtcac gaaaagttaa tcctggtccc agcccagcct cttagtagct |
| 3121 | gtgaggcttt gcacaacttc cttgtcttgg aaagttatat aaacctcatc tgcaagaaaa |
| 3181 | ggcatgatac tgattgcaaa aatggcagca actcttcatc ccttcctgta tttatgccct |
| 3241 | ttgtgatgtg cttttatagc tgctttcatt gagaggcaga atctgtttcc caaaccttga |
| 3301 | atctcactgg ccttatttgc tctggcagta gaaagctgtg aatatgacca tgtgccagtt |
| 3361 | tggggccgag gcacaaaagc tcttgaatgc ttctgctttt tatttcacaa ccctgtcatc |
| 3421 | tccataataa caagtccatg ttagcctgct agaggatgag atactatagg gaggaaaacc |
| 3481 | aaagtgccac agttgacagg cagctcaccc tcagaagcag cattgcctag tcaaccagca |
| 3541 | gctgaccaca cgtgcctgaa agggcccagc agagaccaga agaatggctc acttgagccc |
| 3601 | agcctagaag gtggaccagc tcaatctaat acgttttggg gaattttact gtgaagcaat |
| 3661 | aggtaatacg tacacctagt gcagacagga tgccaggatt caatgacatg ttgattgcaa |
| 3721 | tttaccaggc aaacacaagg caccctgtat gtactgattt ccttttccct ttatgtaaag |
| 3781 | ttggatttct ggttagacgg tgttgagcca tacagaagtg cacgtagaca tgcatgtgat |
| 3841 | ttgtgcttaa actcacacac ttaaattcca caccacagga ggaaacagaa aaccacagcc |
| 3901 | tgaccattag ggccaaagcc aaattgcaaa atgagaagtt tgtctttaat gtattccctc |
| 3961 | tacatctaaa gtcctgaggt taaggctgtg gacaaatctg aagacatggg ttttcttctc |
| 4021 | ctggggcccc tcatcttttt ttcactgtct tatctataga agaggctggg cccctggcaa |
| 4081 | gagcaggtgg gaaatagtag actccctcta gatacttctt tattgcagcc cacttctctc |
| 4141 | tctgggcact gtctctgcca cccttgggcc tgaggagaga tgttagcaga aggagggcat |
| 4201 | tccctcacct ctagcagaaa agaggggttc gggcccttca ctccctgagg tcaggggaac |
| 4261 | cagagccctc aaaactgcac ttcagtgtga cagtcattgc ccacaggggt ctacttagca |
| 4321 | cctagaatgt ggctggtctg aatcggtaca tgctggaagt ttaaaacgca gagtgacttt |
| 4381 | gaaagattta gtaccaaaaa acagaacaac aaaaaatact tcattaatat tatatttatc |
| 4441 | atatatttaa attataatat tttgtatata ttgagttaaa taaagtattg caatcaattt |
| 4501 | tacctgttta ttcctcgttt ctgttgctac tagaaatttt aaaatgacac ctgtggctca |
| 4561 | catgtgattt ctgtggaaat cgcagccttg gaggattgct tcccttttca aaactcgggc |
| 4621 | tcctcagact gcttcctcag aagaccagag gccatgaagg ttaataagtc tgaaatttta |
| 4681 | aaagaattat tattatattg tttcctattt taaatatcat atatattatt cacaaatgaa |
| 4741 | tgtaacattg tatacaaggt taagtgtata taaatgaatg tcccctaggc aaggctgggt |
| 4801 | cccaacccag agaggaaacc ctcttggaga agagctgtag ccagaacgct gcctcattca |
| 4861 | gacacgcgcg ggagctgtca cctgtcacta agagtctgag ggggcggggc ctgaggccct |
| 4921 | acccaatcag gggcaccggg gcggggactg cctaatccgg cgctccgcta gaaaacccga |
| 4981 | ggcggcttcc gggtgtgccc ggcctttgtc tctcgtgccc gcacgtgcgt gtctcggtca |
| 5041 | gtagccctgc gcttctcctt cactctcggc ggttcaggag gctctgccgc agccggggcc |
| 5101 | ctcctgtgac ctgcatgtac tgggggattc gcagggagga tgtcgggaca ccccggaagc |
| 5161 | tgggaaatgg tgagttgcgc cgccgggggt ctggagaagg gaaggggctg tggcggcccc |
| 5221 | gggccccggg ctcccagcgg tcggctcccg agggcggacc cgggtccctg ctggcgcggc |
| 5281 | gcggcgcggc cctcggtcct ttctggcgcc cggcagagct gggcgggcag cggcaccccg |
| 5341 | ggctccggtc ccgtctctgc accgcgactt ccgtcctgcc cgaagcagct tccctccccg |
| 5401 | gcccgcgccc gcagccccgc ggctcctcgg gatcctgcag tgagacccga ggcgcctcag |
| 5461 | gggagaggcc cagcgcggtg tgcgggctcg cgcctgcgag gagctgtggc cactggcgtt |
| 5521 | tccagtccct ccttaatcct gttaaaaatt aaactgaggt ttaattaaag cgttacagag |
| 5581 | tttgagcaaa cagcgattca tgagtagggg agcacccagc cacggttttt ggtttgtggg |
| 5641 | ccaccggacg gtcttgatgg aaaggcttct gtaaagtgtg taaggaaaca aaccaaattt |
| 5701 | atttattttt tattttaatg atcgggtggg gcgggggggg ggtctccctg tgttgcccag |
| 5761 | gctggtcttg aacttctggt ctcaacctcc cgaagtgctg ggattacccc gcccagccca |
| 5821 | aagcaacccc aatttaataa ctgattggtt ataattatgt agttctgtta ttcggattat |
| 5881 | ttaggtggat ggttcctggt tatgtaatca gaggtaaatt gctgatttgg ggttaagttt |
| 5941 | tctttccctt caagttaatt acaagaaagg catgaagtac ctttaggttt tcttggtgag |
| 6001 | gaactcaggg caccatcatc acttaaggga ggagaccacc cctcatattg tcttatgccc |
| 6061 | aatttctgcc tccaaagaaa gaagaagtaa aaactaaaag gcagaaatga aatccacagg |
| 6121 | cagacagccc ggcgccgcgt cctgggcctg gttacagatt gacccttgac ctgaccggtt |
| 6181 | gtgttatcta tagattccag acattgtatg gaaaagcact gtgaaaatcc ctgtcctgtt |
| 6241 | ctgttccgtt ctgattgccg gtgcatgcag cccccagtta tgtaccccct gcttggtcaa |
| 6301 | tcgatcacga ccctctcacg tggaccccct tagagttgta agcccttaaa agggacagga |
| 6361 | attgctcact tggggagctg ggtttttgga gacgtgagtc ttgccaaagc ttccggcaga |
| 6421 | atgaagccct tccttcttta actcggtgtc tgaggggttt tgtctgcagc ttgccctgct |
| 6481 | acaacttcag tctaatttct tcctatttaa ttatttccac aagggtactg gtttcccctg |
| 6541 | catttcccaa atgtaaggca agcagggtct caaatccatg accgtattcc ccagcctaac |
| 6601 | ttctagaact tgcagtaaaa ttctaaattt ccagtttctt cctcatactc tcaaactcca |
| 6661 | acttcacctc tccaatttac aacattatta attatttgtt ctttattgtg catttgaacc |
| 6721 | agatgcagta atgtaattgt ttatcatgtt ttcactgagc agtgaggagc tgtttctgaa |
| 6781 | cattccttgt gaaaacagag aataatcacc tgatctagtc cactataaaa aattctttgt |
| 6841 | acctctcctc cttttgtctt ccctaggcac actcacaaac gtctttggat ggaggtttcc |
| 6901 | ctttggaacc ttcacgtggt gttgtgtcct cagccaccct cctgtctttt ctgggtcctg |
| 6961 | ggttcaaaac tgtttgagga tgacctaatg tgcccacact ggccctgtct cttggagtgt |
| 7021 | cataaatagt gaatgtcagc tgttgggtcc tcttctccca gaagacaagg tgaggtttag |
| 7081 | gggtggagcc tctcagagga gcagctggat ggcatgaatc ccctgggacc gagaggagtc |
| 7141 | tcctgggata ctcttctata ctcttcctct aaaaagctaa tccactagga cttagatttt |
| 7201 | tttttcttct cttacccccg ttatcactcc ttagagacaa aatgctggtc aggcagtcgg |
| 7261 | atactggtat tgagggaaga aagaatttct gccctctgga ttgtctcatg cttgtgaagg |
| 7321 | gagaacagct attccaaagt acaaggaaac cccgccccgc agtgaggcag gaacctgaaa |
| 7381 | agcaaaatgc atttggggca cagtggggac acggcacagg tttctggggg agggtgggca |
| 7441 | ttgagtactt ccctgagcag gatggggtgg gaggacacag tggggacact gggagagaca |
| 7501 | ctggtgtcat ccaatccaat gctggtattt tgagggagag acagaaatga ttcctgccct |
| 7561 | ctggattctt tcacacttgt gaagggagaa aaacaacctt aaagaacaag aaaatttctt |
| 7621 | cccaggaaga tgacacaaaa acctgccaag caaactgcac cttaggcaca cagtgggcca |
| 7681 | cagtgcattg cgctgggagg ttggtccttg agtacgtggt ctctccaatg aggagaccgg |
| 7741 | ggaccaggga atctcctgct tgagagatgg tcttacttga catgtgagtc aaacatatct |
| 7801 | gtgttccagt tagcgctgct cctccctggg ctgtcctctt gcaaagcttt gttcatttat |
| 7861 | tggtgcctta ggtaaacata aaatgtattt catcaataga gctttaaaga taaaatattt |
| 7921 | acaaagaggc aaagaaagaa gtggatattt taaatatatg tagatatttt aaatccactt |
| 7981 | tatatatata tttagttttt taattaaaaa aatttttaat gttatatcaa tacatttggg |
| 8041 | gtagcaggtg gtttttggtt acaaggataa gctctctagt ggtgattgct gagattttgg |
| 8101 | tcctgtcacc ttatcagtgt acgctgtacc caatatgcag tctcaccccc tcccaccctt |
| 8161 | gcccccaaat caccaaggtc cattatatca ttcttatgcc tttgtaacct catagcttag |
| 8221 | ctcccactta taagtgagaa catagaatat ttggttttcc attcctgagt tacttcactt |
| 8281 | agaataatgc ctccatttcc atccaagttg ctgcaaagcc cattatttca ttccttttta |
| 8341 | tggctgagta gtattccacc acattttctt tatcccctca ttggttgatg ggcattttag |
| 8401 | gtttgtttca tatttttgca gtggcaaatt gtgctactgt taacatgcgt gtgcaagtgt |
| 8461 | ttttgtttgt ttgtttttta attgagacag agtctcactc tgttacccag gctgtagtgc |
| 8521 | agtggcacga tcttggctca ctacaacctt ccgtcgcctg ggttcaaggg attctcatgc |
| 8581 | cttagcctcc caagtagctg ggactacagg catgcaccat cacgcccagc taaatttttt |
| 8641 | ttgtattttt agtagagaca gggttttgcc atgttggcca ggctggtatc gaactcctga |
| 8701 | tgtcaggtta tccacctgcc tcagcctccc aaagtgctgg gattgcaggc atgagccacc |
| 8761 | acgcccagcc gtgttttttt catataatga cttcttttcc tttgggtaca tacccagtag |
| 8821 | tggcattgct ggattgaatg gtagttctac ttttagttct ttaaggaatc ttcatactgt |
| 8881 | tttccatggc ggttgtacta gttgacactc ccatcagcag tgtagaagtg ttccctgttc |
| 8941 | accacatcca caccagcatc ccagcatcta ttgtttttta ttttatgatt atggccattc |
| 9001 | ttacaggagt aaggtggtat ctcattatgg ttttaatttg catttccctg atagtgatgt |
| 9061 | tgagcatttt ttcatattta ttggctattt gtatatcttc ttttgagaac tattcatgtc |
| 9121 | ctttgcctac ttttttgatg gaattttttc ttgctgattt gagttttttg tgaattctgg |
| 9181 | atattagtcc tttgtaggat gcatagttgg tgaagatttt ctcccactct gtgggttgtc |
| 9241 | tgtttactct gctgattatt tcttttgttg tgcagaagct ttttaattta attaggtgcc |
| 9301 | atttgttcat ttttgttttt gtgcatttgc ttttgggttc ttggtcatga gttctttgcc |
| 9361 | caagccaatg tttagaagag tttttttgat atattttatt ttcattcccc tgagtgtatg |
| 9421 | tggtaacttt ttgaggtctc gtctcttctt ttgggtagtt tcaaatgaaa ttctagggct |
| 9481 | tttaatcagg aatgctacca gggaagagaa ataggaaaaa tctctcttcc tttatgatta |
| 9541 | cagaaagtga atacatttcc acacacaaaa gtgtggtaca taaattggtg aattacaaaa |
| 9601 | attaaccaaa acatcagttt gtttcctgca ggatgaagaa tttgttagag tgggtaagtc |
| 9661 | tgtgctgttt tctgttgcct gcatttggca cattaatgct aaatctgtac agcaaccttg |
| 9721 | cccgatcact gtggccagga tttccaatac tgtgttgaat gggagtgata aaagcagagg |
| 9781 | ttctctaccg ctctttcctg atcttgggag aaaagggttt ttttgcttgt ttgttttggt |
| 9841 | atttcagcat tgatggtgat gttagtgctg gtttttcata ctgtagtttc cttgtagtcc |
| 9901 | tagtttattg tgtattttta tcatgagtgg gtcttaataa tttgccaaat acttttcctg |
| 9961 | catccgttgc aatagctacg atatttcagt cattttgcaa atgtcataca ttacactgat |
| 10021 | tgatttttca tatattgaac cattttattt tagggataaa tctcacttgg ttttggtgta |
| 10081 | taatcctttt actgtgctgc taaatatggc ttgtgagtac ttaagtgagg atttctgcgt |
| 10141 | tagtatgatg ctaaatatgg tttgttggta tttaagtgag gatttctgca ttaatagtta |
| 10201 | taaagaacat ttgtagcttg ttgtagtgtc tgtccaattt aggggttctg attactacat |
| 10261 | caatgtcata taactgtgag tctgtttaga ttttctaatt cttcatgatt cattcttggt |
| 10321 | acactgtgtg tttctagaca tttgttcacc tcatcaatgt tatccaactt gctggtgtgc |
| 10381 | ccttagagac tttttacata agatttaaga cacagatttc ttttatatgt gttttactct |
| 10441 | tattgtatat gtattctgct tattttgtaa atgtttattt taggttcagg ggtacatgta |
| 10501 | cacaatagca aatacatgga atcaacctaa atgcccatca acagtagact ggacaaagaa |
| 10561 | aatgtggtat atacacattt gtgaataatg ctacagtgaa cctatgcatg catatttctt |
| 10621 | tatggtagaa tgatttctat ttcttcggat atatactcaa taaatgggat tgctaggttg |
| 10681 | aatgggagtt ctgttttaat ttctttgaga aatcaccaca ctgctttcca aaatgagtga |
| 10741 | attgaattcc caccagcagt gtataagcat tcccttttct ccacaacctt accagcatct |
| 10801 | ggttttgttt tgttttgttt tcactttatt atttttttga ggcagtgtct tgctctgtca |
| 10861 | cccaggctgg agtgcagtgg cacgattttg gcttactgca gcctctgcct cctgggctca |
| 10921 | agcagtcatc ccacctcagc ctcacgagta gctgggacta tgggcatgtg ctaacaggcc |
| 10981 | cggctaattt ttttattctt tcttagagac aatgtctcac tatattgtcc aagctggtct |
| 11041 | cgaactcctg gactgaaata atcctcctgc ctcaacctcc tgaactgctg ggattacagg |
| 11101 | catgaaccac cctgcccagg ctattttttg agtttttagt aacagcatta tgactggtat |
| 11161 | aagatggtat ctcatcgtgg ttttaatttg catttcttta gtgatgagtg ataatgttca |
| 11221 | tttttctcat gtttgtatat cttttgaaaa gtgttcatgt cctttgccca ctttttaatg |
| 11281 | tttcattctt gtttcctctt tgcttgttaa tttatttaag tttcttacag attctggata |
| 11341 | ttagaccctt gttagatgca tagtttgcag atattttctc tcactttgta ggttctctgt |
| 11401 | ttaatcccgt tagtttgttt tgctgtgaag aagttcttta gtttaattat gtcccatttg |
| 11461 | tcaacttttt cttttgtggc agtcgctttg ggcatcttca tcatgaaata cttgccaggt |
| 11521 | cctatgtcca gaatggtatt tcctaggtta tcttccaggg tttttttatc tttaaatttt |
| 11581 | tacatttaag tctttaatgc atctaaagtt tatatttgta tatggtgtaa ggaaggggtc |
| 11641 | cagtttcaat cttctgcatg tggctagcca gtaatcctag caccatttat cgaataggaa |
| 11701 | gtcctttcct cattatttgt ttttgttgat tttgtcaaaa atcagatggt tggccaggcg |
| 11761 | cagtggctca cgcctgtaat cccagcactt tgggaggctg aggcaggtgg atcatgaggt |
| 11821 | caggagatcg agaccatcct cgctaacacg gtgaaaccct gtctctacta aaaatacaaa |
| 11881 | aaattagcag ggcggggtgg caggcgcctg tagtcacagc tactcgggag gctgaggcag |
| 11941 | gagaatggca tgaaccgggg cagcggagct tgcagtgagc cgagattgtg ccactgcact |
| 12001 | ccagcctggg tgacagagca agactccgtc tcaagaaaaa aaaaaaaatc agatggtcgt |
| 12061 | aggtgtgtgg cattatttct gggctttcca ttctgctcca tcagtctgtc tgtttttata |
| 12121 | ccattaccat gctgttttgg ttacagtagc tttgtagtat agtttgaagt tggttaatat |
| 12181 | gatgcctcca aactttgttc ttcttgcttg gctatgtggg atctttattg gttccacatg |
| 12241 | aattttaaaa tagttttatc taatttggtg aacaatgtaa ttggtagttt gataggaata |
| 12301 | gcattgaatc taaattgctt tgggtagttg gaccatttta gcaatattaa ttcttcctat |
| 12361 | caatgagcat ggaatgtttt tccattcatt tgtatcatct ctgtttgttt tgttttgttt |
| 12421 | tttttttccc agcagcatct tgtaactcat tgtagagatc tttcatctcc tggttagcta |
| 12481 | tattcctaga tattttattc tttttgtggc tattgtgaat gggattgcct tcctgatttg |
| 12541 | gctctctgca tggatattat tcgtgtatag aaatgctcct aatttttgta catttatttt |
| 12601 | ctgtcctgaa actgtgctaa agttgtttat caggagcttt tcaacagaga ctgtggggtt |
| 12661 | ttggggggta taggatcata tcatttgcaa acagagaagt ttgatttcct gtcttcctat |
| 12721 | ttggatgcat tttatttcag tagtatgtta ataggagtgg taaaagaggg catccttgcc |
| 12781 | ttgttccagt tctcaagagg aatgtttcca gctgttgcct attcactatg atgttggctg |
| 12841 | tgggtttttc atagatagct cttattattt tgaagtatgt ttctccgatg cctagtttgt |
| 12901 | tgagggtttt taacatgaag ggatgttgaa atttatttaa agcttttttt tgcatctatt |
| 12961 | gagatgatca tgttgttttt ttgtttttag ttttgtttat gttgtgaatc acatttattg |
| 13021 | acttgtatat attgaaatgt tacatccaag ggataatgcc tacttaatca tggtggtgga |
| 13081 | ataggttttt ggtgtgtgct ggatttggtt tgctagtatt ttacatctgt ggatttttca |
| 13141 | tctatgttca tcaaggatat tggcctgaag ttttcttttt cattgttgta tgcctgccat |
| 13201 | gtgttgtatg cctgccagga tgacgctggc tgtcatagaa tgagttaagg agtttctcct |
| 13261 | ctttagtttc ttagaatagt ttcagaagga atggtaccag ctcttcttaa tacatctggt |
| 13321 | agaatttagc tgtgaaacca tctggtcctg gcttttctgt ttgataggtt tattattact |
| 13381 | gattaaattt cagagctcat ttttctgttc agggattcca tttcttcctg gttcaatctt |
| 13441 | gggaggtttt ttgtttccag gaatgtattc gtttttctag gttttctagc ctgtatgcat |
| 13501 | agagatgttt gtaataatag cctctgaggg attttttttt tttttagttt ctgtgagtct |
| 13561 | ggtggtaata ccctttttgt cacttctgat tgtgtttata tggatcttct ctccttttct |
| 13621 | ttattagtct agctagcaat ctatcttatt tcaaaaaatt gactcctgaa gttgttgatc |
| 13681 | ttttgctttt ttttctctct ctcttcaatt tatctctgat tttggttttt ttgtcttctg |
| 13741 | ctagctttgg ggttggtctg ctcttgtttc tttagttcct ctaagtgtaa tgttaggttg |
| 13801 | ctaatttgag atcttttcaa agtttttgat gtgggcattt agtgctataa aatttcctct |
| 13861 | taacactgcc ttagctgtgt tccagagatt cttgtatgtt tgttcttatt agtttcaaat |
| 13921 | aatttcttgg tttctacctt aatttcattt ttttcttttg atttgttgag gattgtttta |
| 13981 | tggctgaatg tgtggttgat tttagagtat gtgccatgtg cagctgagaa gaatgtatat |
| 14041 | tctgttgttt tttggtgaag agttctgtag gtgtccatta ggtccatttg gtgaagtgtt |
| 14101 | gagttcagat ctcaaatatc tttgttagtt ttctgccttt atgatctaat accatcagtg |
| 14161 | gggcattgaa gcctcccact gttaactctg tggttaagtc tctttgtagg tgtgtaagaa |
| 14221 | tttgccgtat gaatctgagt gctcttgtgt tgggtgcata cgtatttagg atagttaggt |
| 14281 | ctgcttgttg gattgaacct tttaccaata tgtaataccc gtgtcttttt ttgtttttgg |
| 14341 | tttaaagtct gttttatctg aaatttgact agtaatccct gctttttttt ttttttttgc |
| 14401 | caagggttaa tatattttat atataagtaa agatatacag tcatacattg cttaatgtgt |
| 14461 | tgttaggcag ttttatagtg agaacataat agcgtgtact tacgcaaacc tagatggcat |
| 14521 | aatataccac acacctaggc tatatggtat ggcctattgc tcctagattt acttttttct |
| 14581 | gttttccatt tgtgttgtag atttttctct atctctttac tttgaatgtg tgggcgtcat |
| 14641 | tgcatgtgag atgggtctct tgaagacagc atacgattgg ttttgtttct ctatccagct |
| 14701 | tgtcattttg ccttttaatt ggggcattta gcccatttac attcaaggtt actattcata |
| 14761 | tgtgtagatt tgatcttgtt attgtgtcgt tagctagtta ttatgcagac ttgtttgtat |
| 14821 | gcttgcttta tagtgtcact ggtctatgta cttacgtgtg tttttgtggt ggctgttaat |
| 14881 | gatctttcca tatttagcac tacctttagg aactcttata aggcagatct agtgggaaca |
| 14941 | aattctctta gtagttgctt gtctgaaaat gattatgaag cttcttttgg ctggatggga |
| 15001 | aattcatggt tggaatttct tttccttaag aatgctgaat ataaactctg ataatgcaca |
| 15061 | ataaattttc tggacattag agaaaaatta ctggactgta gattgtggta aagttcaaat |
| 15121 | tactggaaat agcaatggag tatatccact gccttggcct tcatttctgc gtatttattt |
| 15181 | ggtgtggtaa tcaccacttg agattctcta atttttgttc ttctaaagaa tgtggactat |
| 15241 | tatttactat tattattata gtaatatgcc atattatttg gctgttttat taccaagaaa |
| 15301 | atggatctct ttccataaat ggtctttttt cttgttatgt atcttttgct tttttttccc |
| 15361 | attttttaat catgttttca ttcattttta ctgtgcacac taatttcttt tatattgcat |
| 15421 | gtattgcaga tttttatttt actgtttttt gtcttttaat tctcctaatt ttaatatatt |
| 15481 | tgaatttgtc agtctctatt gtttgctcct tttctcctat tcatatgtca aaaaaaacct |
| 15541 | tgtctgtgac ttctgcaagt ggtctattta tactttttac ttttgaatat atgacaaatt |
| 15601 | gattattgtc caactaatag aggtcaatgc aattcatttt tttcccaatt aaataccggt |
| 15661 | tagctttgtt ccatgtttaa agtgcttgat acttccttca ctatcctaca atgcctcctc |
| 15721 | tgttattagt cagctttcca tatcaattcc agctaattaa agactatatg cctttctctt |
| 15781 | ggcctagttc tttatctctc tgcttatact acactgccct gaatattata tttgaaaatc |
| 15841 | attgatgtgt attaggcaaa ccatcacact gttattcaga agactttgac ggttgttagg |
| 15901 | cgtttgttat ttctgtaggt tttgaaaagt agtttctgct gaactgctag aaagtctgtt |
| 15961 | tcaggcatct atgaaatttt acataaatat catttgtaag aggtgacatc tagatgacat |
| 16021 | tgagtttttt tctttgaacg catgcctttt cacttattct attctgcttt gacatttttc |
| 16081 | agtagaaatt gtaaaataat tatttttgga ttattgaata ttgattatta gtgttaatta |
| 16141 | cctcttgatt aaatttgggc ttgagtgaag cacttataag cttctacaca ttttctcttt |
| 16201 | gtgtaaccac gtggattggg ctcaatttcc caattaaaaa gttgtgacat catatatgaa |
| 16261 | atattatcta ccagggaagc tcatcagata ctcagtgctc agagtgtgtt tttgtttgtt |
| 16321 | tattgaggct gatcccccta ggcaccactg ctacaataaa acaaaataat agacttttgg |
| 16381 | aaggaaaaca ggggttcagc aaataccaga ttgtttagat aaactattta gcaacagtga |
| 16441 | gctactttta tcacttaggt tgttgggatg cctcttaaaa tgtaagttcc ctcataccag |
| 16501 | caaaggttga actttgtagc aggtatttct aagggtaagt agtcttgtga cggctcatat |
| 16561 | taactctctt atgcacacca gatagtaaga ctttgatatg atatgtactg caggtatatg |
| 16621 | ctataaagaa aaatcataac agcaaatatt actccctgga cccaccaaca agaagggtgt |
| 16681 | tccttctctg ctgctgcctt gctgaacagt tcaactttag cataagaact gctggctgag |
| 16741 | cgcggtggct cacgcctgtt atcccagcac tttgggaggc caagacaggc agatcacgag |
| 16801 | gtcaagagat cgaggccatc ctggccaaca tggtgaaatc ctgtctctac taaaaataca |
| 16861 | aaaattagct gggcgtggtg acatgtgcct gtagtcccag ctacttgaga ggcagaggca |
| 16921 | ggagaatcac ttgagcctgc gaggcgaaga ttgcagtgag ctgagacggt gccactgcac |
| 16981 | tccagcctgg caacagagtg agactccgtc tcaaaaaaaa aaaaaaaaaa aaaaaaagaa |
| 17041 | ctgctatgtg ggagaaagga agtgtagaca gagtcatagt tctagtgacc aaatagtaga |
| 17101 | ataaatgttc gccacagaat cagatgaaca tcttataaat tgactacaga tttccagtgc |
| 17161 | tttcagtctc atccatcctc tacaaaaatg tggaatattt tagaactcgg ttgcctttga |
| 17221 | ggatgtggct gtgaacttca cccaggagga gtgggctttg ttggatcctt cccagaagaa |
| 17281 | tctctataga gacgtgatgc aggaaacctt caggaatctg gcttccatag gtaagaatca |
| 17341 | caatatttct tcacttagtc aatcagaaag taagtgtttc atggttttca atgtcattcc |
| 17401 | atgatttgga atgtacaaag ggaacacttt gatgagtgaa tgaggcatgg acccagtgtg |
| 17461 | caatgactct tgtttcttag tctatttttt aattgtgata acagaatacc tgatcctggg |
| 17521 | taatttacaa aaatagtttt atattggttt atgattctag tggctagaaa gtccaaggtt |
| 17581 | gcatctggta aggacctgat gctacttcag ctcagggcat gaagcagaag caagtgggca |
| 17641 | tgtgcaaagg gatcacagca agaaaataag agcaattctt gggagctaaa cttgctttta |
| 17701 | taacaacctg ccctctggca actaattcag tcttgcaaga gtaagagaaa acatgcattc |
| 17761 | ccaagcaagg acattaatct attaataagg aatctgtacc cataacacaa acaatgttgc |
| 17821 | catattgatg accaaattac agcattcatt ttggtgggta caaaccatat ccccaacata |
| 17881 | gcacctagag tctagtaatt tttctataat ttcaaataat ttgtaatata tttctgggtc |
| 17941 | tacattttag gaaacaaagg ggaagaccag agcattgaag atcagtacaa aaattcttca |
| 18001 | agaaatctaa ggtaatttgc cctcacaaga ggaagcagtc ccccttgggg tcaattgtag |
| 18061 | tatataatca tatgtttaaa gcaaacaagg ccaggcatgg tggctcacac ctgtaatccc |
| 18121 | agcactttgg gatgctgagg tgggcggatc acctgaggtc aggagtttga gaccagcctg |
| 18181 | accaacacgg caaaaacccg tctctactaa aaatacaaaa ttagcccagt gtgatggtgc |
| 18241 | atgcctgtaa tccctgctgt ttgggaggct gaggcaggag aattgcttga acctgggagg |
| 18301 | cggaggttgc aatgagctga ggttgtgcca ttgcactcca gccagggaaa caagagcaaa |
| 18361 | acaccatctc ggggaaaaaa aaaaaccaag caaacaaaat aaattaagac aacatcaatt |
| 18421 | tattctcaaa gtttttacca aaaatatata cttcaatgta acatggatgt taaatgtttg |
| 18481 | caaaatagtt cacttgaaaa cagtattgtt ttagtctgct tgcattgcta taaaggaata |
| 18541 | cctaggctga gtaatttata aggaagagtt ttatttggct aatggttctg caggctttat |
| 18601 | cagaagcata aagccagcat ctgcttctga tgaggacctc agagagcttc cagtcatggc |
| 18661 | agaaggggaa ggggagctga catgtcacat ggggagagaa ggagcaagag agagagaaag |
| 18721 | gaagtgccaa agtttttggg gtttttttgt tttgttttgt tttgtttgag atggagtctc |
| 18781 | tctctggtat ccagactgga gtgcagtggt gcagtcttgg ctcactgcaa cctccgcctc |
| 18841 | ccgggttcaa gtgattctcc ggactcagtc tcccaagtag ctgggattac aggaacatgc |
| 18901 | caccacaccc agctaatttt tgtattttta gtagagacag catttcacca tgttggccag |
| 18961 | gctggtcttg aattcctgac ctcaagtgat ctgcccacct tggcctccca aagtgctggg |
| 19021 | attacaggcg tgagccacca tgcccgacca tttttttttt ttttttaaca atcggatctt |
| 19081 | tcaggaacta atagagcgag aagtcactca ttaccacaac agtgccactt atgtggaatc |
| 19141 | tgctcccatg acccaagcac ctcacaccag gtcatacctc caacatgagg atcaaatttc |
| 19201 | agcatgaaat ttgaagggag aaaataccca aactgtattc aatactaaga aactgcatat |
| 19261 | gagaatatta ctgtattgtt aatagctata ggggagggag ccatgttgta gactaatcaa |
| 19321 | tccatttatg ttcaatttgt ttatgttaga aaacctgcac tttctctgat attggtagca |
| 19381 | gtgtaagttc agacttagta ataaaagaaa ataactaata aaccattaat gatggggttg |
| 19441 | tcattttttg cagaagtcat atgatagaca tactgtgtaa aattaaataa gtcagtgtag |
| 19501 | aaaaacctcc agatgccaaa ttttaatctg aacaaaaaaa ttcctgctag agtaaaacca |
| 19561 | catgaatgca ttgtgtgtga aaaattcttc atacgtcatt catcccttca taggcacatc |
| 19621 | atatctcatt ctggaaacaa cccatatggg tgtgaggaat gcggaaagaa gccatgtaca |
| 19681 | tgtaaacaat gtcagaaaac ttccctttct gtcacaaggg ttcacagaga cacagtaatg |
| 19741 | cacactggaa atggacatta tggttgtaca atatgtgaga aagtttttaa tattccca [symbol] t |
| 19801 | tcatttcaga tacatcagag aaatcacact ggagagaaac cctatgaatg tatggaatgt |
| 19861 | gggaaagcct taggtttttc ccgttctctt aatagacata aaaggattca cactggagaa |
| 19921 | aaacgctatg aatgtaagca atgtgggaaa gccttcagtc gttccagtca ccttcgtgac |
| 19981 | catgaaagaa ctcatactgg agagaaaccc tatgaatgta agcactgtgg gaaagccttc |
| 20041 | cgttactcca attgccttca ttaccatgaa agaactcaca ctggagagaa accttatgtg |
| 20101 | tgcatggaat gtggcaaagc tttcagttgt ctcagttcct tgcaaggaca tataaaggct |
| 20161 | catgctggtg aagaacccta tccatgtaag caatgtggga aagccttcag atacgccagt |
| 20221 | tcccttcaga aacacgagaa aactcatatt gcacagaaac cctatgtatg taacaattgt |
| 20281 | ggtaaaggct tcagatgttc cagttccctt cgtgaccatg aaaggactca tactggagag |
| 20341 | aaaccctatg aatgtcagaa atgtggcaaa gcctttagtc gtgctagtac cctttggaag |
| 20401 | cataaaaaaa ctcatactgg agaaaagccc tataaatgta aaaaaatgta aaggctttaa |
| 20461 | tcactacagt ttttgtcaaa aacatgaaca gtcacatact tgagagaaac tgtgaatgta |
| 20521 | aggtgtagga aagtacttaa ttttcccaga tttcctcaaa tacatgaaac gaatcaaact |

The mutation c.219-1G>- of the above-mentioned ZNF124 gene can also be described as the deletion of G at the 19799th base of the ZNF124 gene of the above-mentioned sequence.

In addition, the mutation is not detected in 3000 control samples. Based on this, there are sufficient reasons to believe that the homozygous deletion mutation c.219-1G>- which occurs in the ZNF124 gene is an important pathogenic factor for retinitis pigmentosa diseases, and therefore, early screening or diagnosis of retinitis pigmentosa diseases can be achieved by detecting the mutation c.219-1G>-.

The present disclosure provides a detection reagent for detecting ZNF124 gene mutation for use in early screening or auxiliary diagnosis of retinitis pigmentosa diseases.

The present disclosure provides a method for early screening or auxiliary diagnosis of retinitis pigmentosa diseases in a subject, comprising: detecting the mutations of the ZNF124 gene in the subject's sample.

In one or multiple embodiments, the mutations of the ZNF124 gene comprise c.219-1G>-.

In one or multiple embodiments, the detection of the ZNF124 gene mutation in the sample of the subject is carried out by using a detection reagent.

In one or multiple embodiments, the sample is the body fluid, tissue or hair of the subject.

In one or multiple embodiments, the subject is a human.

In one or multiple embodiments, the body fluid is selected from blood, saliva or semen.

In one or multiple embodiments, the ZNF124 gene mutation c.219-1G>- is a homozygous mutation.

In one or multiple embodiments, the detection reagent is selected to be applicable to any one of or a combination of the following methods for detecting ZNF124 gene mutations: a restriction fragment length polymorphism method (PCR-RFLP), denaturing gradient gel electrophoresis (DGGE), allele-specific PCR, a DNA sequencing method, a DNA chip detection method, a time-of-flight mass spectrometry (TOF) and a single-strand conformation polymorphism analysis (SSCP).

In optional embodiments, the DNA sequencing method is a whole exon sequencing method or a Sanger sequencing method.

It should be explained that there are many methods or techniques for detecting gene mutations in this field, which comprise, but are not limited to, the restriction fragment length polymorphism method (PCR-RFLP), denaturing gradient gel electrophoresis (DGGE), allele-specific PCR, the DNA sequencing method, the DNA chip detection method, time-of-flight mass spectrometry (TOF), single-strand conformation polymorphism analysis (SSCP) and the like, as mentioned above, and on the basis of the gene sequence and the type of the mutation to be detected as disclosed in the present disclosure, a person skilled in the art can easily design corresponding detection reagents for detecting the above-mentioned mutations, regardless of what detection reagent is used for detecting ZNF124 gene mutations or what detection method it is suitable for, it falls within the scope of protection of the present disclosure as long as it is used for early screening or diagnosis of retinitis pigmentosa diseases.

The embodiment of the present disclosure provides a reagent or a kit for early screening or auxiliary diagnosis of retinitis pigmentosa diseases, which contains the detection reagent for detecting ZNF124 gene mutations.

In one or multiple embodiments, the mutations of the ZNF124 gene detected by the detection reagent comprise c.219-1G>-.

In one or multiple embodiments, the detection reagent is selected to be applicable to any one of or a combination of the following methods for detecting ZNF124 gene mutations: a restriction fragment length polymorphism method, denaturing gradient gel electrophoresis, allele-specific PCR, a DNA sequencing method, a DNA chip detection method, time-of-flight mass spectrometry and single-strand conformation polymorphism analysis.

In one or multiple embodiments, the DNA sequencing method is a whole exon sequencing analysis or a Sanger sequencing method.

In one or multiple embodiments, the ZNF124 gene mutation is a mutation on exon 4 of the ZNF124 gene.

In one or multiple embodiments, the ZNF124 gene mutation is a homozygous mutation on exon 4 of the ZNF124 gene.

In one or multiple embodiments, the retinitis pigmentosa diseases comprise night blindness and/or impaired visual field.

The embodiment of the present disclosure provides a reagent or a kit for early screening of retinitis pigmentosa diseases, which contains the detection reagent for detecting ZNF124 gene mutation.

By detecting the ZNF124 gene mutations, the reagent or kit for early screening provided by the present disclosure can be used for early screening of retinitis pigmentosa diseases before marriage or before childbirth, which helps to prevent offspring from suffering from retinitis pigmentosa diseases.

In optional embodiments, the mutations of the ZNF124 gene detected by the detection reagent comprise c.219-1G>-.

In optional embodiments, the detection reagent is selected to be applicable to any one of or a combination of the following methods for detecting ZNF124 gene mutation: a restriction fragment length polymorphism method (PCR-RFLP), denaturing gradient gel electrophoresis (DGGE), allele-specific PCR, a DNA sequencing method, a DNA chip detection method, time-of-flight mass spectrometry (TOF) and single-strand conformation polymorphism analysis (SSCP).

In optional embodiments, the DNA sequencing method is a whole exon sequencing analysis or a Sanger sequencing method.

The embodiments of the present disclosure provide a reagent or a kit for auxiliary diagnosis of retinitis pigmentosa diseases, which contains the detection reagent for detecting ZNF124 gene mutation.

By detecting the mutations of the ZNF124 gene, the reagent or kit for auxiliary diagnosis of retinitis pigmentosa diseases provided by the present disclosure can provide auxiliary basis or reference materials for the diagnosis of retinitis pigmentosa diseases and thus provide guarantee for accurate diagnosis of retinitis pigmentosa diseases.

In optional embodiments, the mutations of the ZNF124 gene detected by the detection reagent comprise c.219-1G>-.

In optional embodiments, the detection reagent is selected to be applicable to any one of or a combination of the following methods for detecting ZNF124 gene mutation: a restriction fragment length polymorphism method (PCR-RFLP), denaturing gradient gel electrophoresis (DGGE), allele-specific PCR, a DNA sequencing method, a DNA chip detection method, time-of-flight mass spectrometry (TOF) and single-strand conformation polymorphism analysis (SSCP).

In optional embodiments, the DNA sequencing method is a whole exon sequencing analysis or a Sanger sequencing method.

In optional embodiments, the type of the sample detected by the reagent or kit is body fluid, tissue or hair from the subject to be tested.

In optional embodiments, the subject to be tested is a human.

In optional embodiments, the body fluid is selected from blood, saliva or semen.

It should be explained that the types of the samples detected by the reagent or kit for early screening or auxiliary diagnosis of retinitis pigmentosa diseases provided by the present disclosure comprise, but are not limited to, blood, saliva and semen, and other types of samples containing the DNA from the subject to be tested can also be used as test samples for the reagent or kit of the present disclosure.

The detection reagent for detecting ZNF124 gene mutation as used in the present text comprises various reagents used when detecting the ZNF124 gene mutation. For example, the detection reagent for detecting the ZNF124 gene mutation comprises the reagent used in PCR-RFLP, such as PCR primers; for example, it comprises reagents (such as solvents) required to extract samples; for example, it comprises reagents used in DGGE; for example, it comprises primers or labeling reagents that may be used in the DNA sequencing. The detection reagents for detecting ZNF124 gene mutation may also comprise reference substances and standard substances, for example, a wild-type ZNF124 gene and its nucleotide sequence, and for example, the c.219-1G>-mutant of the ZNF124 gene and its nucleotide sequence described herein. A person skilled in the art can select detection reagents for detecting the ZNF124 gene mutation according to needs, for example, according to the detection method or sample type.

The characteristics and performance of the present disclosure will be further described in detail below in conjunction with embodiments.

### Example 1

Family Analysis and Discovery of ZNF124 Mutation

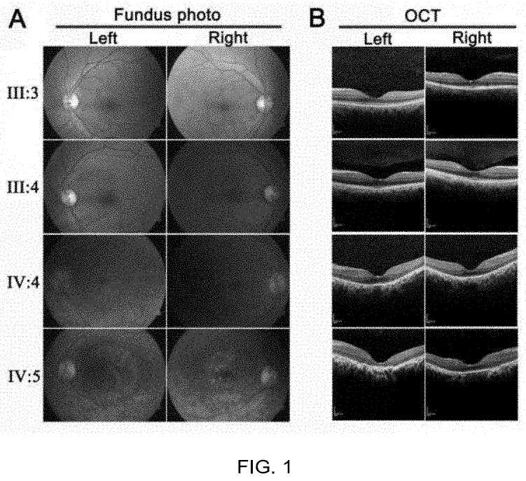

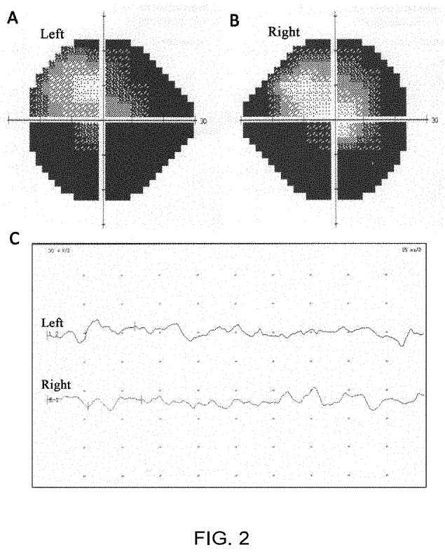

Among the collected RP families, the RP074 family is a Han ethnic family in southwest of China with autosomal recessive retinitis pigmentosa, and has 13 people of 3 generations, and 2 people have RP in total. The clinical examination showed that the proband patients IV:4 and IV:5 of the RP074 family had weak night vision from the age of 15, and gradually developed to night blindness with impaired peripheral visual field. Fundus examination revealed that the papilla of optic nerve atrophied in both eyes of the patients, and revealed retinal artery vascular stenosis and retinal pigmentation (A in FIG. 1); optical coherence tomography (OCT) of retina showed that the patient's retinal neuroepithelial layer became thinner, with the outer nuclear layer being the most obvious; the reflection of the connection layer of the inner and outer segments of the photoreceptor disappeared, and part thereof was discontinuous; the pigment epithelial cells were partially lost and thinned, and the pigment epithelial layer atrophied (B in FIG. 1), which was a typical pathological characterization of RP. Visual field examination revealed that the patient's peripheral visual field was damaged (A, B in FIG. 2). ERG examination revealed that the function of the patient's photoreceptor cells was impaired (C in FIG. 2).

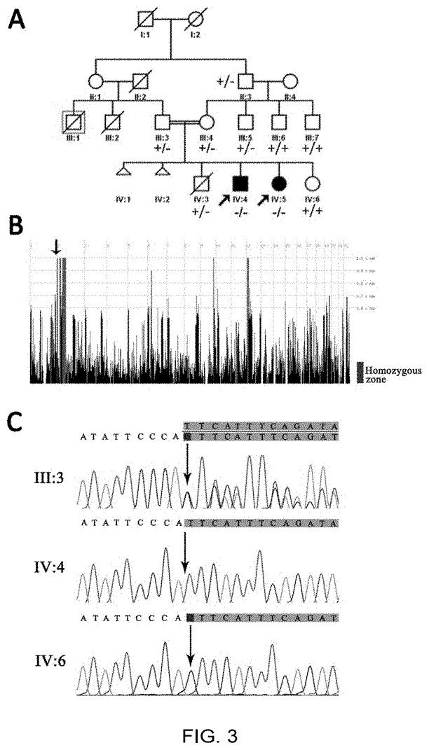

By the whole exon sequencing analysis for the proband patients IV:4, IV:5 and their parents III:3 and III:4 in Shanghai Renke Biotechnology Co., Ltd. (A in FIG. 3), it was found that the ZNF124 gene had a homozygous deletion mutation (NM_003431:exon4:c.219-1G>-) co-separated with the disease. The analysis of the homozygous region of the genome also revealed that there was a homozygous region at the end of chromosome 1 (shown by the arrow B in FIG. 3). The sequencing of the DNA of the proband IV:4, IV:5 and their parents through the Sanger method verified and showed that their parents carried heterozygous mutation, and the proband had homozygous mutation (C in FIG. 3), and the genotypes of the rest of the family members were shown in A of FIG. 3, which was in line with typical arRP genetic characteristic.

### Example 2

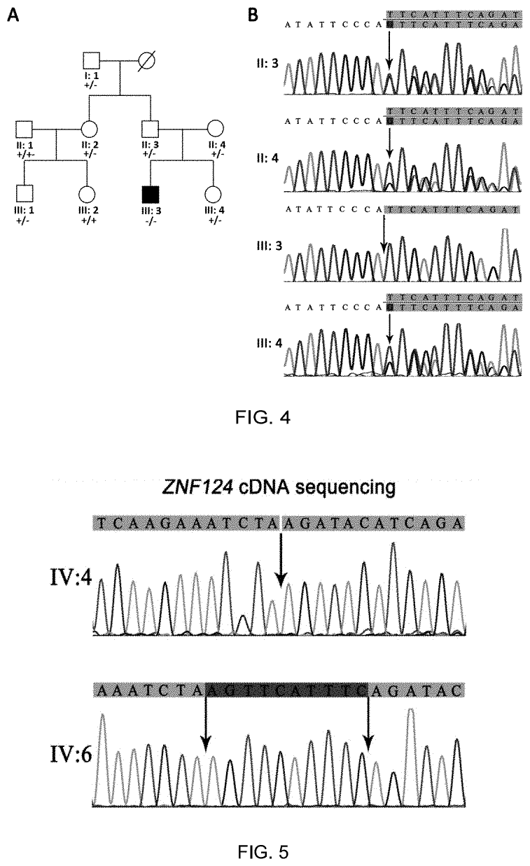

Further data analysis was conducted to the collected whole-exon-sequencing RP samples in which known gene mutations were not detected, and it was found that another autosomal recessive RP family RP131 carried the ZNF124 mutation. RP131 was a Han RP family in northern China (A in FIG. 4). The proband carried a homozygous mutation of the ZNF124 (B in FIG. 4), and his parents were heterozygous carriers. The proband was a male patient with night blindness at the age of about 20 and progressive visual field atrophy.

This mutation site was not detected in dbSNP database, 1000 Genomes, NHLBI Exome Sequencing Project (ESP), the ExAC Browser (Beta), gnomAD database, and was a rare mutation. The mutation was not detected in 3000 healthy control samples. This splicing mutation led to the base deletion of the encoding region after the 73^{rd} codon, resulting in a frameshift mutation; and the mutant ZNF124 protein only retained the N-terminal KRAB-A box domain, and these data indicated that this ZNF124 mutation was a relatively good RP candidate gene mutation. The detection of this gene mutation could be used for early screening or auxiliary diagnosis of retinitis pigmentosa diseases.

### Example 3

In order to detect the influence of the deletion mutation (NM_003431:exon4:c.219-1G>-) on the transcription and translation of ZNF124 protein, the peripheral blood RNA of the RP074 proband and their parents was extracted and reversely transcribed into cDNA, the method was as follows:

(a) peripheral blood leukocytes were separated and placed in a 1.5 ml centrifuge tube, 1 ml Trizol extracting solution was added, and the resultant was left at room temperature for 20 minutes;

(b) 200 μl chloroform was added and mixed fully, and the resultant was left undisturbed at room temperature for 10 minutes;

(c) the sample was placed in a 4-degree centrifuge and centrifuged at 10,000 rpm for 15 minutes;

(d) the supernatant was carefully aspirated, an equal volume of isopropanol was added and mixed fully, and the resultant was centrifuged at 10,000 rpm to precipitate RNA;

(e) the precipitated total RNA with 75% ethanol was washed, the resultant was centrifuged and precipitated again, then air dried, and DEPC water was added to dissolve; and

(f) the extracted total RNA was used to synthesize to cDNA with a cDNA synthesis kit (Invitrogen, Waltham, Mass., USA). Primers were designed based on the cDNA sequence of Gm20541:

| (SEQ ID NO. 2) | |
|---|---|
| ZNF124-cDNA-F: | 5′-tgaggatgtggctgtgaact-3′; |
| and | |
| (SEQ ID NO. 3) | |
| ZNF124-cDNA-R: | 5′-actggaacgactgaaggctt-3′. |

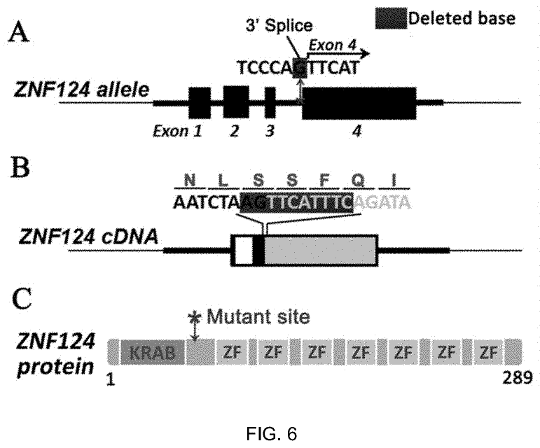

Then Sanger sequencing was conducted to the cDNA segment of ZNF124. The results showed that a segment deletion with a total of 10 bases of AGTTCATTTC after the mutation site appeared in the cDNA of the patient's ZNF124, which caused a base frameshift after the deletion site (FIG. 5), and resulted in an error in the encoding of the ZNF124 protein (FIG. 6).

### Example 4

Study on the Expression of ZNF124 Tissue Cells

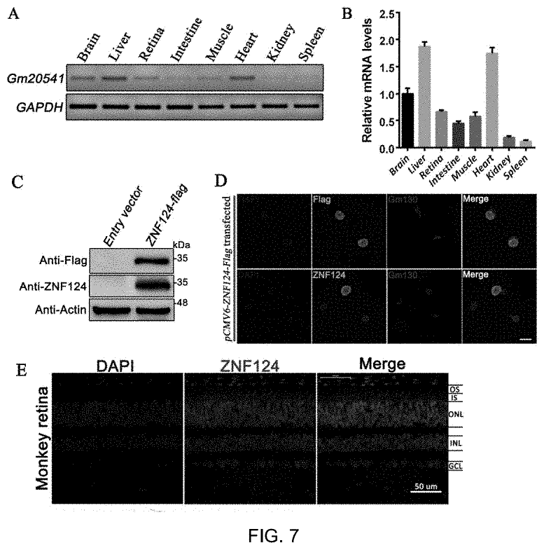

RT-PCR experiments of various mouse tissues showed that the homologous gene Gm20541 of ZNF124 in mice was widely expressed in the brain tissue, retina, liver, heart, and muscle and the like of a mouse (A and B in FIG. 7). In order to further explore the cell expression position of the ZNF124, we purchased a ZNF124 (Proteintech) polyclonal antibody, and transfected constructed pCMV6-ZNF124-Flag expression plasmid into 293T cells with Lipofectamine 3000 transfection reagent (Thermo Fisher Scientific). The total cell protein was collected 48 hours after transfection for western blot detection, and the results showed that the antibody could specifically recognize the ZNF124 protein expressed by 293T cells after transfection of pCMV6-ZNF124-Flag plasmid, which matched the position of the tag antibody (C in FIG. 7), which proved its antibody specificity was better. The ZNF124 expression plasmid was transfected into COS7 cells again, and the ZNF124 and its tag Flag were immunohistochemically stained, respectively, and the results showed that the ZNF124 protein was located in the nucleus (D in FIG. 7). Immunohistochemical staining experiments on the retinal sections of rhesus monkey showed that the ZNF124 protein was located in the nucleus of retinal photoreceptor cells, bipolar cells and ganglion cells (E in FIG. 7), which suggested that it could act as a transcription factor to regulate the expression of other genes.

### Example 5

Constructing Gm20541 Gene Knockout Mice and Studying the Retinitis Pigmentosa of Gm20541 Gene Knockout Mice

In order to further study the relationship between ZNF124 and retinitis pigmentosa, the inventors constructed mice, in which Gm20541 gene in the mice was knocked out, wherein the Gm20541 gene was homologous to the ZNF124 gene, and determined the retinitis pigmentosa of the gene knockout mice. Although this experimental example studied retinitis pigmentosa in Gm20541 gene knockout mice, a person skilled in the art would easily understand that as the mice were a representative animal of mammals, when Gm20541 gene or its homologous gene, for example, ZNF124, was knocked out from other mammals, they should also have similar phenotypes. That is, the Gm20541 gene could be used as a representative of the homologous gene ZNF124 to study the association between the Gm20541 and the retinitis pigmentosa in a mouse model.

(A) the Specific Operation of Gm20541 Gene Knockout

Steps to Obtain Gene Knockout Mouse:

step 1, the 5′ arm, the expression frame containing the reporter gene GFP, the expression frame containing the NEO resistance gene, the third exon with loxP sites aligned in the same direction at both ends and the 3′ end arm homologous to the Gm20541 gene of the mouse were cloned into a BAC vector to replace the third exon of the Gm20541 gene to be knocked out;

step 2, a DNA homologous recombination technology was used to replace the third exon in the Gm20541 gene to obtain mouse embryonic stem cells which Gm20541 gene is conditionally knocked out;

step 3, the embryonic stem cells obtained in step 2 was used to prepare a chimeric mouse containing the Gm20541 gene knockout cells; and

step 4, the chimeric mouse obtained in step 3 was mated with a wild-type mouse, and heterozygous mice with the Gm20541 gene knockout were screened from the offspring.

Identification

The experiment result of the first filial generation of positive mice was identified by the long-distance PCR, the primer pair of Gm 5′LRF and SA3′R were used to amplify the long arm at the 5′ end, and the amplification product was 2.3 Kb.

(SEQ ID NO. 4)

| | |
|---|---|
| Gm 5′LRF: | 5′-GGCAGGATCTTCACCTGTTGACCAACATGCCT-3′; |
| and | |

(SEQ ID NO. 5)

| | |
|---|---|
| 5A3′R: | 5′-CCAACCCCTTCCTCCTACATAGTTGGCAGT-3′. |

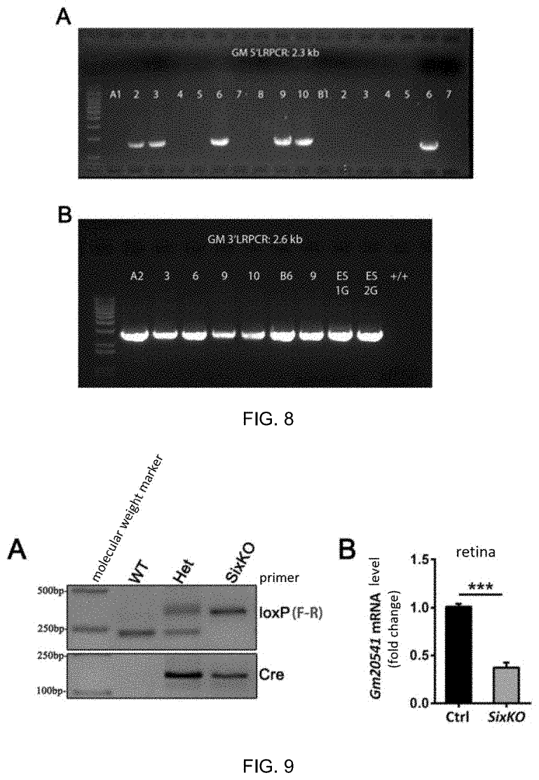

Referring to A in FIG. 8 for the results, the amplification product was 2.3 Kb, wherein A2, 3, 6, 9, 10 and B1 were positive.

The long arm at the 3′ end was amplified by using the primers of:

(SEQ ID NO. 6)

| | |
|---|---|
| NeoF: | 5′-CGCCTTCTTGACGAGTTCTTCTGA-3′; |
| and | |

(SEQ ID NO. 7)

| | |
|---|---|
| Gm 3′LRR: | 5′-GGTGCTTGAGTAGTGTTGAATCTCAGTGGACCA-3′ |

Referring to B in FIG. 8 for the results, the amplification product was 2.6 Kb, wherein A2, 3, 6, 9, 10, B6, 9 ES1G and ES2G were positive heterozygotes, and +/+ was the wild type control.

Step 5, the heterozygous mouse animal obtained in step 4 was mated with the transgenic mouse, i.e., the FLPer mouse, to obtain heterozygous mice with Gm20541 gene conditionally knocked out;

step 6, the heterozygous mice with Gm20541 gene conditionally knocked out obtained in step 5 were mated with each other to obtain homozygous mice with Gm20541 gene conditionally knocked out; and

step 7, the homozygous animal with Gm20541 gene conditionally knocked out obtained in step 6 was mated with the Six3-Cre gene transgenic animal to obtain mice with Gm20541 gene knocked out in retinal precursor cells.

The transgenic mouse, the FLPer mouse, was purchased from Czech Forest Laboratories, USA (strain name: B6.12954-Gt(ROSA)26Sortm1(FLP1)Dym/RainJ). Six3-Cre transgenic mice (MGI: 3574771) were presented by the Anderson Cancer Center, University of Texas in the USA. Six3 is a landmark transcription factor for ventral forebrain and retinal precursor cells, which specifically drives the expression of Cre gene in retinal precursor cells, Cre protein could enter the nucleus, identify LoxP sites on the genome, and achieve gene knockout.

Steps to Identify Gene Knockout Mice

To identify the genotype of the above mouse whose Gm20541 gene was knocked out from the retinal precursor cells, the operation was as follows:

(1) a little tissue sample was cut from the tail of the mouse and placed in a 1.5 ml clean centrifuge tube;

(2) 100 μl of lysate (40 mM NaOH, 0.2 mM EDTA solution) was added to the centrifuge tube, and was heated in a metal bath at 100° C. for 1 h, (3) the centrifuge tube was taken out, and cooled to room temperature, 100 μl of a neutralization solution (40 mM Tris-HCl, pH5.5) was added and the resultant was centrifuged at 10000 g for 2 min, and the supernatant was taken for mouse genotype identification.

(3) PCR amplification: the PCR reaction system was configured according to the following system:

2×Taq Mix: 10 μL

tail tissue lysate: 2 μL

primer 1 (Gm20541-loxP-Forward or Six3-Cre-Forward): 1 μL (concentration: 10 mM)

primer 2 (Gm20541-loxP-Reverse or Six3-Cre-Reverse): 1 μL (concentration: 10 mM)

ddH₂O: 6 μL.

The primer sequence was as follows:

Gm20541-loxP-Forward sequence:
(SEQ ID NO. 8)
5′-ATTCCCCTTCAAGATAGCTAC-3′;
Gm20541-loxP-Reverse sequence:
(SEQ ID NO. 9)
5′-AATGATCAACTGTAATTCCCC-3′;
Six3-Cre-Forward sequence:
(SEQ ID NO. 10)
5′-GCCGCCGGGATCACTCTCG-3′;
and
Six3-Cre-Reverse sequence:
(SEQ ID NO. 11)
5′-CCAGCCGCCGTCGCAACTC-3′.

Amplification procedure: after the PCR reaction system was prepared, preheating on a PCR machine at 95° C. for 5 minutes was conducted to fully denature the template DNA, and then an amplification cycle was started. In each cycle, a temperature of 95° C. was held for 30 seconds to denature the template, then the temperature was lowered to a renaturation temperature of 58° C., and held for 30 seconds to fully anneal the primer and the template; a temperature of 72° C. was held for 30 seconds to make the primer extended on the template to synthesize DNA, so as to complete a cycle. This cycle was repeated 25 times to accumulate a large amount of amplified DNA fragments. Finally, a temperature of 72° C. was held for 5 minutes to allow the product to extend completely, and the product was stored at 4° C.

(4) Gel electrophoresis

1 g agarose was weighed and put in 100 ml TAE buffer, after it was melted in a microwave oven, it was made into 1% agarose gel. 10 μl PCR product was taken to the sample hole, and agarose electrophoresis was conducted at a constant voltage of 120V for 15 min, and it was imaged with a gel imaging system.

The upper part of A in FIG. 9 was the identification result of conditional knockout of the Gm20541, WT represented the wild-type control, and the band size was 223 bp; Het represented heterozygote with two bands of 223 bp and 358 bp; SixKO represented homozygote with a band size of 358 bp. The lower part of A in FIG. 9 was the identification result of Six3-Cre. The size of Six3-cre was 200 bp. According to the result of A in FIG. 9, it was shown that the adopted identification method could effectively identify the genotype of newborn mice. In the above, the homozygous mice with Gm20541 gene knocked out could be used as a retinitis pigmentosa disease model (hereinafter, SixKO or cKO is used to denote homozygous mice with Gm20541 gene knocked out; Ctr refers to a wild-type; Het refers to heterozygote), and verification of related phenotypes was performed.

(5) The verification of the gene knockout efficiency in the retina of Six3-cre knockout mouse was analyzed by RT-PCR experiment, and the method was as follows:

(a) the retina tissues of the wild-type mouse and the mutant mouse were separated, respectively, and were placed in a 1.5 ml centrifuge tube, 1 ml Trizol extracting solution was added and the resultant was then left at room temperature for 20 minutes;

(b) 200 μl chloroform was added and mixed fully, and the resultant was then left undisturbed at room temperature for 10 minutes;

(c) the sample was placed in a 4-degree centrifuge and centrifuged at 10,000 rpm for 15 minutes;

(d) the supernatant was carefully aspirated, an equal volume of isopropanol was added and mixed fully, and the resultant was centrifuged at 10,000 rpm to precipitate RNA;

(e) the precipitated total RNA was washed with 75% ethanol, and the resultant was centrifuged to precipitate again, then air dried, and DEPC water was added to dissolve;

(f) the extracted total RNA was synthesized into cDNA by using the cDNA synthesis kit (Invitrogen, Waltham, Mass., USA). Primers were designed based on the cDNA sequence of Gm20541:

| (SEQ ID NO. 12) | |
|---|---|
| Gm20541-cDNA-F: | 5′-TCGGTCTCATCTTCATTCCC-3′; |
| (SEQ ID NO. 13) | |
| Gm20541-cDNA-R: | 5′-GGAAGGCTCTGTTCCGGTAT-3′; |

and

(g) the extracted cDNA was used as a template to perform RT-PCR. Electrophoresis was conducted after amplification.

The results were shown by B in FIG. 9, and it could be seen that the expression of Gm20541 in the retina of the homozygous mice with Gm20541 gene knocked out was significantly reduced, which was detected by the RT-PCR method. In B of FIG. 9, Ctrl referred to the wild-type control, SixKO referred to the Gm20541 gene knockout homozygous mouse; Gm20541 RNA level referred to the relative change in RNA expression level, with the wild-type expression level being 1.

(B) Vision Test

ERG vision detection was conducted to 4-month-old Gm20541 gene knockout homozygous mice.

1. Scotopic animals should adapt to darkness all night, and there should be absolutely no light in the environment.

2. The mice were narcotized the next day, weighed, and given intraperitoneal injection, and deep anesthesia was recommended.

3. The animals were fixed and subjected to mydriasis: after the anesthesia was completed, the mouse was fixed on the front of an animal test platform with tape under illumination of dark red light: it was necessary to ensure that the mouse lay on its stomach, i.e., with respect to the stimulation port of a flash stimulator, both eyes were at the same height, fully exposed, and dropped with mydriatic agent.

4. Electrode installation: after preheating an electroretinograph (Espion Visual Electrophysiology System, DiagnosysLLC, Littleton, Mass., USA), a conductive paste was applied on ear clip electrodes, the tail was clamped, and the “ground” interface of an amplifier was inserted; a double-ended needle electrode was inserted into the posterior neck skin (approximately between the two ears), and the “negative” interfaces of two channels were connected at the same time; a gold ring electrode was clamped on an electrode holder of the animal experiment platform, and its angle was carefully adjusted. The top of the center of the cornea was slightly touched. The positive electrode of channel 1 was connected to the right eye, and the positive pole of channel 2 was connected to the left eye. Physiological saline was dropped in both eyes through a needle tube to improve the contact effect between the gold ring electrode and the cornea. It was ensured that the two gold ring electrodes touched the same position on the center of the cornea of the two eyes at the same angle and in the same way.

5. The oscillometric signal was recorded and after it was confirmed that everything was correct, the dark red light was turned off. An attempt may be made first to record the ERG detection at a scotopic light intensity of 0.003 cd·s/m² to confirm the quality of the signal: if there was a big difference between the amplitudes of two eyes, which were different from expectation, it was recommended to check the installation position of the gold ring electrodes again. Then, the signals at scotopic light intensities of 0.3/3.0/20.0 cd·s/m² were recorded sequentially. It should be noted that the system would automatically turn on the background light after the scotopic light intensity detection of 20.0 cd·s/m² was completed. Likewise, the timer should be turned on, and record was made after adaptation to light for 10-15 minutes.

6. After adaptation to light, light adaptation of 3.0 cd·s/m² and 20.0 cd·s/m² were sequentially recorded.

The waveform under light intensity was recorded, and the evoked potential of flashing retina under 20.0 cd·s/m² light intensity was finally recorded.

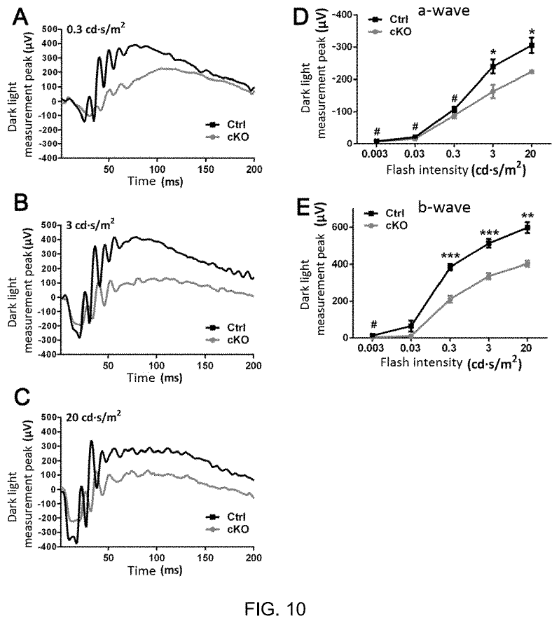

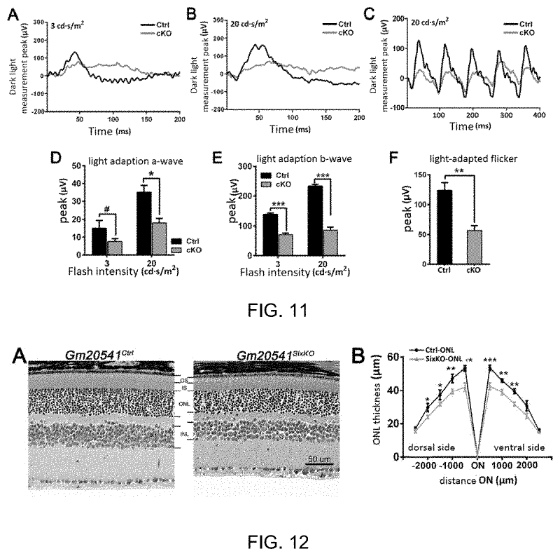

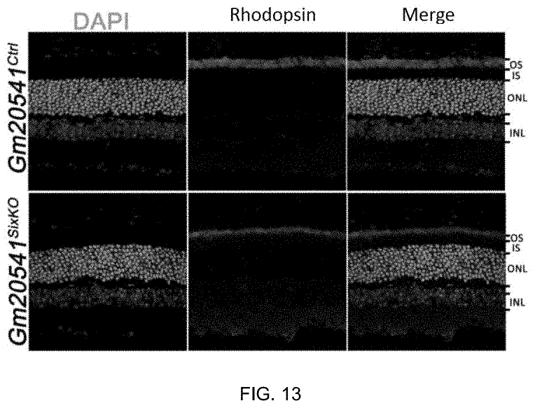

The results showed that at 4 months, compared with Ctrl (control) mice, the a-wave and b-wave of cKO (Gm20541 gene knockout homozygous mice) mice were significantly reduced under both scotopic and light-adapted conditions, which indicated that after Gm20541 was knocked out, the vision was impaired (referring to FIGS. 10 and 11).

(C) H&E Staining for Paraffin Sections of Retina

Paraffin section and staining by the hematoxylin-eosin staining method (H&E staining method) were performed on the retina of 4-month-old mice. The specific operation was provided as follows.

1) The eyeball tissue of the mouse was quickly taken and was placed in a fixative and fixed for 24 h.

2) The resultant was embedded by the paraffin and sectioned with a thickness of 4 μm.

3) The sections were routinely deparaffinized with xylene and washed with multi-grade ethanol and then washed by water: xylene (I) for 5 min→xylene (II) for 5 min→100% ethanol for 2 min→95% ethanol for 1 min→80% ethanol for 1 min→75% ethanol for 1 min→distilled water to wash for 2 min.

4) Hematoxylin staining was conducted for 5 minutes, and rinsing with tap water was conducted.

5) Hydrochloric acid ethanol differentiation was conducted for 30 seconds. 6) Soaking in tap water was conducted for 15 minutes.

7) The resultant was placed in eosin solution for 2 minutes.

8) Conventional dehydration, vitrification and section sealing: 95% ethanol (I) for 1 min→95% ethanol (II) for 1 min→100% ethanol (I) for 1 min→100% ethanol (II) for 1 min→xylene carbolic acid (3:1) for 1 min→xylene (I) for 1 min→xylene (II) for 1 min→neutral resin to seal.

9) Pictures under the microscope were taken.

It was found that at 4 months, compared with Ctrl (control) mice, the outer nuclear layer of the retina of SixKO mice began to become thin, which indicated the death of photoreceptor cells (FIG. 12).

(D) Immunostaining of Frozen Sections of Retina

The 4-month-old Gm20541 gene knockout homozygous mouse obtained from (A) was sacrificed by breaking its neck, then the eyeballs were quickly taken out and put in 4% PFA, after fixing on ice for 15 minutes, the cornea was cut, and then was continuously fixed on ice. 2 h later, they were rinsed with PBS buffer for 3 times, then the eyeballs were put in 30% sucrose solution for dehydration for 2 h, then the cornea and crystals were cut off under a dissecting microscope, and embedded in OCT and quickly placed in the refrigerator at −80° C. to be frozen. After about 10 minutes, the OCT-embedded eyeballs were taken out, equilibrated in a freezing microtome at −25° C. for about 30 minutes, and then they could be sliced. The thickness of the section was 12 μm.

After the slicing was completed, sections of higher quality were selected and placed in an oven at 37° C. for 30 min; then an immunohistochemical pen was used to draw a circle on the area with retinal tissue, and they were washed with PBS three times to remove OCT; and then they were closed and permeated with 5% NDS (containing 0.25% Triton) for 2 h, the primary antibody was incubated at 4° C. overnight. On the next day, after washing with PBS three times, the corresponding fluorescent secondary antibody was incubated, and then they were washed three times with PBS again, and then were sealed and observed.

The results were shown in FIG. 13, when the mice were 4 months old, after staining the outer segment antibody Rhodopsin and inner segment antibody NaK-Atpase with the sections of the frozen tissue of the retina, it was found that compared with wild-type mice, the outer segment of the retina of the Gm20541 gene knockout homozygous mice (SixKO in the figure) was significantly shortened, showing obvious signs of degeneration.

In summary, it can be seen that mice were taken as an example in the experimental example of the present disclosure, through a Cre-loxP gene knockout technology, the Gm20541 gene was specifically knocked out from the retinal precursor cells of the mice. Gene knockout mice showed typical characterizations of retinitis pigmentosa diseases, such as impaired vision, shortened and degenerated outer segments of optic cells, and loss of optic cells. This fully illustrates the significant association between the knockout of Gm20541 gene and retinitis pigmentosa diseases. The Gm20541 gene of the mouse and its homologous gene ZNF124 can be used as markers for retinitis pigmentosa diseases. The detection of this gene mutation can be used for early screening or auxiliary diagnosis of retinitis pigmentosa diseases.

The above-mentioned are only preferred embodiments of the present disclosure and not intended to limit the present disclosure, and for one skilled in the art, various modifications and changes may be made to the present disclosure. Any modifications, equivalent substitutions, improvements and so on made within the spirit and principle of the present disclosure should be covered within the scope of protection of the present disclosure.

### INDUSTRIAL APPLICABILITY

The present disclosure relates to the use of a ZNF124 gene in early screening or auxiliary diagnosis of retinitis pigmentosa diseases. The homozygous deletion mutation c.219-1G>-which occurs in the ZNF124 gene is an important pathogenic factor for retinitis pigmentosa diseases, and therefore, early screening or diagnosis of retinitis pigmentosa diseases can be achieved by detecting the mutation c.219-1G>-.

1-10. (canceled)

11. A reagent or kit for early screening of retinitis pigmentosa diseases, containing a detection reagent for detecting ZNF124 gene mutation.

12. The reagent or kit according to claim 11, wherein the ZNF124 gene mutation detected by the detection reagent comprises c.219-1G>-.

13. The reagent or kit according to claim 11 or 12, wherein the detection reagent is selected to be applicable to any one of or a combination of following methods for detecting the ZNF124 gene mutation: a restriction fragment length polymorphism method, denaturing gradient gel electrophoresis, allele-specific PCR, a DNA sequencing method, a DNA chip detection method, a time-of-flight mass spectrometry and a single-strand conformation polymorphism analysis; and
preferably, the DNA sequencing method is a whole exon sequencing analysis or a Sanger sequencing method.

14. A reagent or kit for auxiliary diagnosis of retinitis pigmentosa diseases, containing a detection reagent for detecting ZNF124 gene mutation.

15. The reagent or kit according to claim 14, wherein the ZNF124 gene mutation detected by the detection reagent comprises c.219-1G>-.

16. The reagent or kit according to claim 14, wherein the detection reagent is selected to be applicable to any one of or a combination of following methods for detecting the ZNF124 gene mutation: a restriction fragment length polymorphism method, a denaturing gradient gel electrophoresis, allele-specific PCR, a DNA sequencing method, a DNA chip detection method, a time-of-flight mass spectrometry and a single-strand conformation polymorphism analysis; and
preferably, the DNA sequencing method is a whole exon sequencing analysis or a Sanger sequencing method.

17. A reagent or kit for early screening or auxiliary diagnosis of retinitis pigmentosa diseases, containing a detection reagent for detecting ZNF124 gene mutation.

18. The reagent or kit according to claim 17, wherein the ZNF124 gene mutation detected by the detection reagent comprises c.219-1G>-.

19. The reagent or kit according to claim 17, wherein the detection reagent is selected to be applicable to any one of or a combination of following methods for detecting the ZNF124 gene mutation: a restriction fragment length polymorphism method, a denaturing gradient gel electrophoresis, allele-specific PCR, a DNA sequencing method, a DNA chip detection method, a time-of-flight mass spectrometry and a single-strand conformation polymorphism analysis; and
preferably, the DNA sequencing method is a whole exon sequencing analysis or a Sanger sequencing method.

20. The reagent or kit according to claim 11, wherein a type of a sample detected by the reagent or kit is body fluid, tissue or hair from a subject to be tested;
preferably, the subject to be tested is a human; and
preferably, the body fluid is selected from blood, saliva or semen.

21-22. (canceled)


SEQUENCE LISTING (13 sequences)

SEQ ID NO: 1 - DNA, 20580 bp, Homo sapiens
tgataggtgcgtgggcccagactccccttttctctgtgattggagtggatgccaagagtgaggagaagccagacagtgggagcgagcacttccaagcctgtgggggtgggggaaccttcctgagccctccagagtgcagagatgcctgggtctgcagccatggcggctgtgcccaggaggatgggcctcctgcctgctccagctcccaagagcacagggatgcccagctcacctatgggtcaacccccaagaacatttataatttctctagaatggcataacccttccaaaagctgacggggaccttgggaatcttcttccagagggtcctgggtcagggatattggcctggctgtgagacagacaacctcctaccctccctgtgcctcctgggccaagacgtggaagcctcatcaggatgcactgccactggcaacaggccaagaacggggtctgcttggggggcgcaggtggttcgggggtcccaggagaattgagaacttcctgcagacaagatgcctctttttcctcatttttggtccaactgatacactggatagtgagataaaccttacattttaatttttatgtattttttcattatatgcaaaatgtatacttaaacagacagtggatttgaagggaaaagtatttgagattcattctttcaagtgtcatttagtcctatatacataggtttatatatattatatatatagtgtatatatatagcatatatagtaatgtatatatgtagtgtatagtatatatgtagtgtatatatactatatatgtgtatatatagtatatatgtatatatgcatacatatgtatatagtatacatgcatacatatgtatatagtatagatacatatataaatatatatgctatatgctatatataggactatatatatatacacatacacacacgcgtatatatatagtcctatatacatagtcctatatacagatttgccctatgtgtaacaactacgtagataaaaaagttatgaaattgtttttggttttacaatgctatattaaaaacattgaaattcaccaattgaacaatctatgcaagggtattatggttttttgttattgttgtttaagtcctgaaatacagagataagagctgagtgagcagccactggtggagaaacagtcttcacggggccagtatttttctgccccgtctcatttggctgcggatcaacacatgtcatttagtacaaacaagcaaacaaaaatagcaatatgcttgtgtgcctaccccagatgctttatgtgcaataatgaaatgaggaaggggaagataagagaatggagaaaaggatactgtaccagccagaatgtggagtcagaatgtggtgtcagaatgtggagtcagaatgtgatgaaatcacattgtgggtttctacatgagaatgtctgctggagttgtaggaagaggtcctgggtaaaggagcaggtgtgttcttagaaggccctcctgtctgctgctggaacacatcacctgtttattcgtcctccatttcccgctttgcaggtgtgtcagtttcactgagtgaccgtccgtaaaatccaaatctgatttgcttcatggccaaaccctgccattcaccataggcttccactccttcattcagtgatgtgtacttcttccccttcaactgcgccccagaaccatcctaccctccaccttcaaattcacagaattgttgttctgtcttgactcaaacttttttcctgccacagcagagcactgaaagtctcttaagttacagcttcacaacagaagtctaaagtctgcgcagaactagcacatctgcagccgtaggagcagcagaatgaccaggcagcagagctttattccatgttttcatctccattcctacaacagcagcggcagcagcaacaactgctactacttctaattgtggtaaaaatacttagaataataatgcataatgacaattgtagttgacatttttggtttatatatgattatttgtaatgttctacaatttacctttagtaatatacacataacttttgcataaacatcaggaccctaaaacaccacacctgatgattgaatctcacctatggacctaaggtcttcacatgctggtgcaggccctctcaccactccatctccaaacagaggatatggtccaccttctgagtcaatatagtagccctgacactcacatgatctatttgaggtcttggaaaacaagcttgccttgttaaaaatcaaaggatgggccgggcacggtggctcacgcctgtaatcccagcactttgggaggccgaggcgggctgatcacgaggtcaggagatggagaccatcctgactaacacgatgaaaccccatctctactaaaaatacaaaaaaattagccgggcatggtggccggcgcctgtagtcccagctactcgggaggctgaggcaggagaatggcgtgaacccgggaggcggagcttgcagtgagccgagatcacaccactgcactccagcctggatgacacagcgagactccatcttaaaaaaaaaaaaaaaaaaaaaaatcaaagaatggcttcaaattgtagtcagctgtttcgttaaattgcagcaggaagaaatttttctcagcaggtctattttcatgtcacttgctttcagagtaagaggttatttcagacacagtggtttgatttagtctttgttgaaaatcataaatagcctacaaacaacaaaaagggatgaataggatttgcgttcatgtggagtgcgtcatctgtgcttttaaatttatcctatgaaaagaaataaaaaatatgtaatatcgaacattggtttcctaaattagcaaatcgaccatttcctcaagttattccacgttcttccaagatgtgtcagaagcattctctggaggcagttgttccaaatggtgaagagtactagatggatcccaatctcacaggattcttgcttctattgcagaagagttactttccaggacttcaaaggtttagagaagctgcttgattcgtgtggcttgcagaggtgcatgggctttggagtcacgaaaagttaatcctggtcccagcccagcctcttagtagctgtgaggctttgcacaacttccttgtcttggaaagttatataaacctcatctgcaagaaaaggcatgatactgattgcaaaaatggcagcaactcttcatcccttcctgtatttatgccctttgtgatgtgcttttatagctgctttcattgagaggcagaatctgtttcccaaaccttgaatctcactggccttatttgctctggcagtagaaagctgtgaatatgaccatgtgccagtttggggccgaggcacaaaagctcttgaatgcttctgctttttatttcacaaccctgtcatctccataataacaagtccatgttagcctgctagaggatgagatactatagggaggaaaaccaaagtgccacagttgacaggcagctcaccctcagaagcagcattgcctagtcaaccagcagctgaccacacgtgcctgaaagggcccagcagagaccagaagaatggctcacttgagcccagcctagaaggtggaccagctcaatctaatacgttttggggaattttactgtgaagcaataggtaatacgtacacctagtgcagacaggatgccaggattcaatgacatgttgattgcaatttaccaggcaaacacaaggcaccctgtatgtactgatttccttttccctttatgtaaagttggatttctggttagacggtgttgagccatacagaagtgcacgtagacatgcatgtgatttgtgcttaaactcacacacttaaattccacaccacaggaggaaacagaaaaccacagcctgaccattagggccaaagccaaattgcaaaatgagaagtttgtctttaatgtattccctctacatctaaagtcctgaggttaaggctgtggacaaatctgaagacatgggttttcttctcctggggcccctcatctttttttcactgtcttatctatagaagaggctgggcccctggcaagagcaggtgggaaatagtagactccctctagatacttctttattgcagcccacttctctctctgggcactgtctctgccacccttgggcctgaggagagatgttagcagaaggagggcattccctcacctctagcagaaaagaggggttcgggcccttcactccctgaggtcaggggaaccagagccctcaaaactgcacttcagtgtgacagtcattgcccacaggggtctacttagcacctagaatgtggctggtctgaatcggtacatgctggaagtttaaaacgcagagtgactttgaaagatttagtaccaaaaaacagaacaacaaaaaatacttcattaatattatatttatcatatatttaaattataatattttgtatatattgagttaaataaagtattgcaatcaattttacctgtttattcctcgtttctgttgctactagaaattttaaaatgacacctgtggctcacatgtgatttctgtggaaatcgcagccttggaggattgcttcccttttcaaaactcgggctcctcagactgcttcctcagaagaccagaggccatgaaggttaataagtctgaaattttaaaagaattattattatattgtttcctattttaaatatcatatatattattcacaaatgaatgtaacattgtatacaaggttaagtgtatataaatgaatgtcccctaggcaaggctgggtcccaacccagagaggaaaccctcttggagaagagctgtagccagaacgctgcctcattcagacacgcgcgggagctgtcacctgtcactaagagtctgagggggcggggcctgaggccctacccaatcaggggcaccggggcggggactgcctaatccggcgctccgctagaaaacccgaggcggcttccgggtgtgcccggcctttgtctctcgtgcccgcacgtgcgtgtctcggtcagtagccctgcgcttctccttcactctcggcggttcaggaggctctgccgcagccggggccctcctgtgacctgcatgtactgggggattcgcagggaggatgtcgggacaccccggaagctgggaaatggtgagttgcgccgccgggggtctggagaagggaaggggctgtggcggccccgggccccgggctcccagcggtcggctcccgagggcggacccgggtccctgctggcgcggcgcggcgcggccctcggtcctttctggcgcccggcagagctgggcgggcagcggcaccccgggctccggtcccgtctctgcaccgcgacttccgtcctgcccgaagcagcttccctccccggcccgcgcccgcagccccgcggctcctcgggatcctgcagtgagacccgaggcgcctcaggggagaggcccagcgcggtgtgcgggctcgcgcctgcgaggagctgtggccactggcgtttccagtccctccttaatcctgttaaaaattaaactgaggtttaattaaagcgttacagagtttgagcaaacagcgattcatgagtaggggagcacccagccacggtttttggtttgtgggccaccggacggtcttgatggaaaggcttctgtaaagtgtgtaaggaaacaaaccaaatttatttattttttattttaatgatcgggtggggcggggggggggtctccctgtgttgcccaggctggtcttgaacttctggtctcaacctcccgaagtgctgggattaccccgcccagcccaaagcaaccccaatttaataactgattggttataattatgtagttctgttattcggattatttaggtggatggttcctggttatgtaatcagaggtaaattgctgatttggggttaagttttctttcccttcaagttaattacaagaaaggcatgaagtacctttaggttttcttggtgaggaactcagggcaccatcatcacttaagggaggagaccacccctcatattgtcttatgcccaatttctgcctccaaagaaagaagaagtaaaaactaaaaggcagaaatgaaatccacaggcagacagcccggcgccgcgtcctgggcctggttacagattgacccttgacctgaccggttgtgttatctatagattccagacattgtatggaaaagcactgtgaaaatccctgtcctgttctgttccgttctgattgccggtgcatgcagcccccagttatgtaccccctgcttggtcaatcgatcacgaccctctcacgtggacccccttagagttgtaagcccttaaaagggacaggaattgctcacttggggagctgggtttttggagacgtgagtcttgccaaagcttccggcagaatgaagcccttccttctttaactcggtgtctgaggggttttgtctgcagcttgccctgctacaacttcagtctaatttcttcctatttaattatttccacaagggtactggtttcccctgcatttcccaaatgtaaggcaagcagggtctcaaatccatgaccgtattccccagcctaacttctagaacttgcagtaaaattctaaatttccagtttcttcctcatactctcaaactccaacttcacctctccaatttacaacattattaattatttgttctttattgtgcatttgaaccagatgcagtaatgtaattgtttatcatgttttcactgagcagtgaggagctgtttctgaacattccttgtgaaaacagagaataatcacctgatctagtccactataaaaaattctttgtacctctcctccttttgtcttccctaggcacactcacaaacgtctttggatggaggtttccctttggaaccttcacgtggtgttgtgtcctcagccaccctcctgtcttttctgggtcctgggttcaaaactgtttgaggatgacctaatgtgcccacactggccctgtctcttggagtgtcataaatagtgaatgtcagctgttgggtcctcttctcccagaagacaaggtgaggtttaggggtggagcctctcagaggagcagctggatggcatgaatcccctgggaccgagaggagtctcctgggatactcttctatactcttcctctaaaaagctaatccactaggacttagatttttttttcttctcttacccccgttatcactccttagagacaaaatgctggtcaggcagtcggatactggtattgagggaagaaagaatttctgccctctggattgtctcatgcttgtgaagggagaacagctattccaaagtacaaggaaaccccgccccgcagtgaggcaggaacctgaaaagcaaaatgcatttggggcacagtggggacacggcacaggtttctgggggagggtgggcattgagtacttccctgagcaggatggggtgggaggacacagtggggacactgggagagacactggtgtcatccaatccaatgctggtattttgagggagagacagaaatgattcctgccctctggattctttcacacttgtgaagggagaaaaacaaccttaaagaacaagaaaatttcttcccaggaagatgacacaaaaacctgccaagcaaactgcaccttaggcacacagtgggccacagtgcattgcgctgggaggttggtccttgagtacgtggtctctccaatgaggagaccggggaccagggaatctcctgcttgagagatggtcttacttgacatgtgagtcaaacatatctgtgttccagttagcgctgctcctccctgggctgtcctcttgcaaagctttgttcatttattggtgccttaggtaaacataaaatgtatttcatcaatagagctttaaagataaaatatttacaaagaggcaaagaaagaagtggatattttaaatatatgtagatattttaaatccactttatatatatatttagttttttaattaaaaaaatttttaatgttatatcaatacatttggggtagcaggtggtttttggttacaaggataagctctctagtggtgattgctgagattttggtcctgtcaccttatcagtgtacgctgtacccaatatgcagtctcaccccctcccacccttgcccccaaatcaccaaggtccattatatcattcttatgcctttgtaacctcatagcttagctcccacttataagtgagaacatagaatatttggttttccattcctgagttacttcacttagaataatgcctccatttccatccaagttgctgcaaagcccattatttcattcctttttatggctgagtagtattccaccacattttctttatcccctcattggttgatgggcattttaggtttgtttcatatttttgcagtggcaaattgtgctactgttaacatgcgtgtgcaagtgtttttgtttgtttgttttttaattgagacagagtctcactctgttacccaggctgtagtgcagtggcacgatcttggctcactacaaccttccgtcgcctgggttcaagggattctcatgccttagcctcccaagtagctgggactacaggcatgcaccatcacgcccagctaaattttttttgtatttttagtagagacagggttttgccatgttggccaggctggtatcgaactcctgatgtcaggttatccacctgcctcagcctcccaaagtgctgggattgcaggcatgagccaccacgcccagccgtgtttttttcatataatgacttcttttcctttgggtacatacccagtagtggcattgctggattgaatggtagttctacttttagttctttaaggaatcttcatactgttttccatggcggttgtactagttgacactcccatcagcagtgtagaagtgttccctgttcaccacatccacaccagcatcccagcatctattgttttttattttatgattatggccattcttacaggagtaaggtggtatctcattatggttttaatttgcatttccctgatagtgatgttgagcattttttcatatttattggctatttgtatatcttcttttgagaactattcatgtcctttgcctacttttttgatggaattttttcttgctgatttgagttttttgtgaattctggatattagtcctttgtaggatgcatagttggtgaagattttctcccactctgtgggttgtctgtttactctgctgattatttcttttgttgtgcagaagctttttaatttaattaggtgccatttgttcatttttgtttttgtgcatttgcttttgggttcttggtcatgagttctttgcccaagccaatgtttagaagagtttttttgatatattttattttcattcccctgagtgtatgtggtaactttttgaggtctcgtctcttcttttgggtagtttcaaatgaaattctagggcttttaatcaggaatgctaccagggaagagaaataggaaaaatctctcttcctttatgattacagaaagtgaatacatttccacacacaaaagtgtggtacataaattggtgaattacaaaaattaaccaaaacatcagtttgtttcctgcaggatgaagaatttgttagagtgggtaagtctgtgctgttttctgttgcctgcatttggcacattaatgctaaatctgtacagcaaccttgcccgatcactgtggccaggatttccaatactgtgttgaatgggagtgataaaagcagaggttctctaccgctctttcctgatcttgggagaaaagggtttttttgcttgtttgttttggtatttcagcattgatggtgatgttagtgctggtttttcatactgtagtttccttgtagtcctagtttattgtgtatttttatcatgagtgggtcttaataatttgccaaatacttttcctgcatccgttgcaatagctacgatatttcagtcattttgcaaatgtcatacattacactgattgatttttcatatattgaaccattttattttagggataaatctcacttggttttggtgtataatccttttactgtgctgctaaatatggcttgtgagtacttaagtgaggatttctgcgttagtatgatgctaaatatggtttgttggtatttaagtgaggatttctgcattaatagttataaagaacatttgtagcttgttgtagtgtctgtccaatttaggggttctgattactacatcaatgtcatataactgtgagtctgtttagattttctaattcttcatgattcattcttggtacactgtgtgtttctagacatttgttcacctcatcaatgttatccaacttgctggtgtgcccttagagactttttacataagatttaagacacagatttcttttatatgtgttttactcttattgtatatgtattctgcttattttgtaaatgtttattttaggttcaggggtacatgtacacaatagcaaatacatggaatcaacctaaatgcccatcaacagtagactggacaaagaaaatgtggtatatacacatttgtgaataatgctacagtgaacctatgcatgcatatttctttatggtagaatgatttctatttcttcggatatatactcaataaatgggattgctaggttgaatgggagttctgttttaatttctttgagaaatcaccacactgctttccaaaatgagtgaattgaattcccaccagcagtgtataagcattcccttttctccacaaccttaccagcatctggttttgttttgttttgttttcactttattatttttttgaggcagtgtcttgctctgtcacccaggctggagtgcagtggcacgattttggcttactgcagcctctgcctcctgggctcaagcagtcatcccacctcagcctcacgagtagctgggactatgggcatgtgctaacaggcccggctaatttttttattctttcttagagacaatgtctcactatattgtccaagctggtctcgaactcctggactgaaataatcctcctgcctcaacctcctgaactgctgggattacaggcatgaaccaccctgcccaggctattttttgagtttttagtaacagcattatgactggtataagatggtatctcatcgtggttttaatttgcatttctttagtgatgagtgataatgttcatttttctcatgtttgtatatcttttgaaaagtgttcatgtcctttgcccactttttaatgtttcattcttgtttcctctttgcttgttaatttatttaagtttcttacagattctggatattagacccttgttagatgcatagtttgcagatattttctctcactttgtaggttctctgtttaatcccgttagtttgttttgctgtgaagaagttctttagtttaattatgtcccatttgtcaactttttcttttgtggcagtcgctttgggcatcttcatcatgaaatacttgccaggtcctatgtccagaatggtatttcctaggttatcttccagggtttttttatctttaaatttttacatttaagtctttaatgcatctaaagtttatatttgtatatggtgtaaggaaggggtccagtttcaatcttctgcatgtggctagccagtaatcctagcaccatttatcgaataggaagtcctttcctcattatttgtttttgttgattttgtcaaaaatcagatggttggccaggcgcagtggctcacgcctgtaatcccagcactttgggaggctgaggcaggtggatcatgaggtcaggagatcgagaccatcctcgctaacacggtgaaaccctgtctctactaaaaatacaaaaaattagcagggcggggtggcaggcgcctgtagtcacagctactcgggaggctgaggcaggagaatggcatgaaccggggcagcggagcttgcagtgagccgagattgtgccactgcactccagcctgggtgacagagcaagactccgtctcaagaaaaaaaaaaaaatcagatggtcgtaggtgtgtggcattatttctgggctttccattctgctccatcagtctgtctgtttttataccattaccatgctgttttggttacagtagctttgtagtatagtttgaagttggttaatatgatgcctccaaactttgttcttcttgcttggctatgtgggatctttattggttccacatgaattttaaaatagttttatctaatttggtgaacaatgtaattggtagtttgataggaatagcattgaatctaaattgctttgggtagttggaccattttagcaatattaattcttcctatcaatgagcatggaatgtttttccattcatttgtatcatctctgtttgttttgttttgttttttttttcccagcagcatcttgtaactcattgtagagatctttcatctcctggttagctatattcctagatattttattctttttgtggctattgtgaatgggattgccttcctgatttggctctctgcatggatattattcgtgtatagaaatgctcctaatttttgtacatttattttctgtcctgaaactgtgctaaagttgtttatcaggagcttttcaacagagactgtggggttttggggggtataggatcatatcatttgcaaacagagaagtttgatttcctgtcttcctatttggatgcattttatttcagtagtatgttaataggagtggtaaaagagggcatccttgccttgttccagttctcaagaggaatgtttccagctgttgcctattcactatgatgttggctgtgggtttttcatagatagctcttattattttgaagtatgtttctccgatgcctagtttgttgagggtttttaacatgaagggatgttgaaatttatttaaagcttttttttgcatctattgagatgatcatgttgtttttttgtttttagttttgtttatgttgtgaatcacatttattgacttgtatatattgaaatgttacatccaagggataatgcctacttaatcatggtggtggaataggtttttggtgtgtgctggatttggtttgctagtattttacatctgtggatttttcatctatgttcatcaaggatattggcctgaagttttctttttcattgttgtatgcctgccatgtgttgtatgcctgccaggatgacgctggctgtcatagaatgagttaaggagtttctcctctttagtttcttagaatagtttcagaaggaatggtaccagctcttcttaatacatctggtagaatttagctgtgaaaccatctggtcctggcttttctgtttgataggtttattattactgattaaatttcagagctcatttttctgttcagggattccatttcttcctggttcaatcttgggaggttttttgtttccaggaatgtattcgtttttctaggttttctagcctgtatgcatagagatgtttgtaataatagcctctgagggattttttttttttttagtttctgtgagtctggtggtaataccctttttgtcacttctgattgtgtttatatggatcttctctccttttctttattagtctagctagcaatctatcttatttcaaaaaattgactcctgaagttgttgatcttttgcttttttttctctctctcttcaatttatctctgattttggtttttttgtcttctgctagctttggggttggtctgctcttgtttctttagttcctctaagtgtaatgttaggttgctaatttgagatcttttcaaagtttttgatgtgggcatttagtgctataaaatttcctcttaacactgccttagctgtgttccagagattcttgtatgtttgttcttattagtttcaaataatttcttggtttctaccttaatttcatttttttcttttgatttgttgaggattgttttatggctgaatgtgtggttgattttagagtatgtgccatgtgcagctgagaagaatgtatattctgttgttttttggtgaagagttctgtaggtgtccattaggtccatttggtgaagtgttgagttcagatctcaaatatctttgttagttttctgcctttatgatctaataccatcagtggggcattgaagcctcccactgttaactctgtggttaagtctctttgtaggtgtgtaagaatttgccgtatgaatctgagtgctcttgtgttgggtgcatacgtatttaggatagttaggtctgcttgttggattgaaccttttaccaatatgtaatacccgtgtctttttttgtttttggtttaaagtctgttttatctgaaatttgactagtaatccctgcttttttttttttttttgccaagggttaatatattttatatataagtaaagatatacagtcatacattgcttaatgtgttgttaggcagttttatagtgagaacataatagcgtgtacttacgcaaacctagatggcataatataccacacacctaggctatatggtatggcctattgctcctagatttacttttttctgttttccatttgtgttgtagatttttctctatctctttactttgaatgtgtgggcgtcattgcatgtgagatgggtctcttgaagacagcatacgattggttttgtttctctatccagcttgtcattttgccttttaattggggcatttagcccatttacattcaaggttactattcatatgtgtagatttgatcttgttattgtgtcgttagctagttattatgcagacttgtttgtatgcttgctttatagtgtcactggtctatgtacttacgtgtgtttttgtggtggctgttaatgatctttccatatttagcactacctttaggaactcttataaggcagatctagtgggaacaaattctcttagtagttgcttgtctgaaaatgattatgaagcttcttttggctggatgggaaattcatggttggaatttcttttccttaagaatgctgaatataaactctgataatgcacaataaattttctggacattagagaaaaattactggactgtagattgtggtaaagttcaaattactggaaatagcaatggagtatatccactgccttggccttcatttctgcgtatttatttggtgtggtaatcaccacttgagattctctaatttttgttcttctaaagaatgtggactattatttactattattattatagtaatatgccatattatttggctgttttattaccaagaaaatggatctctttccataaatggtcttttttcttgttatgtatcttttgcttttttttcccattttttaatcatgttttcattcatttttactgtgcacactaatttcttttatattgcatgtattgcagatttttattttactgttttttgtcttttaattctcctaattttaatatatttgaatttgtcagtctctattgtttgctccttttctcctattcatatgtcaaaaaaaaccttgtctgtgacttctgcaagtggtctatttatactttttacttttgaatatatgacaaattgattattgtccaactaatagaggtcaatgcaattcatttttttcccaattaaataccggttagctttgttccatgtttaaagtgcttgatacttccttcactatcctacaatgcctcctctgttattagtcagctttccatatcaattccagctaattaaagactatatgcctttctcttggcctagttctttatctctctgcttatactacactgccctgaatattatatttgaaaatcattgatgtgtattaggcaaaccatcacactgttattcagaagactttgacggttgttaggcgtttgttatttctgtaggttttgaaaagtagtttctgctgaactgctagaaagtctgtttcaggcatctatgaaattttacataaatatcatttgtaagaggtgacatctagatgacattgagtttttttctttgaacgcatgccttttcacttattctattctgctttgacatttttcagtagaaattgtaaaataattatttttggattattgaatattgattattagtgttaattacctcttgattaaatttgggcttgagtgaagcacttataagcttctacacattttctctttgtgtaaccacgtggattgggctcaatttcccaattaaaaagttgtgacatcatatatgaaatattatctaccagggaagctcatcagatactcagtgctcagagtgtgtttttgtttgtttattgaggctgatccccctaggcaccactgctacaataaaacaaaataatagacttttggaaggaaaacaggggttcagcaaataccagattgtttagataaactatttagcaacagtgagctacttttatcacttaggttgttgggatgcctcttaaaatgtaagttccctcataccagcaaaggttgaactttgtagcaggtatttctaagggtaagtagtcttgtgacggctcatattaactctcttatgcacaccagatagtaagactttgatatgatatgtactgcaggtatatgctataaagaaaaatcataacagcaaatattactccctggacccaccaacaagaagggtgttccttctctgctgctgccttgctgaacagttcaactttagcataagaactgctggctgagcgcggtggctcacgcctgttatcccagcactttgggaggccaagacaggcagatcacgaggtcaagagatcgaggccatcctggccaacatggtgaaatcctgtctctactaaaaatacaaaaattagctgggcgtggtgacatgtgcctgtagtcccagctacttgagaggcagaggcaggagaatcacttgagcctgcgaggcgaagattgcagtgagctgagacggtgccactgcactccagcctggcaacagagtgagactccgtctcaaaaaaaaaaaaaaaaaaaaaaaaagaactgctatgtgggagaaaggaagtgtagacagagtcatagttctagtgaccaaatagtagaataaatgttcgccacagaatcagatgaacatcttataaattgactacagatttccagtgctttcagtctcatccatcctctacaaaaatgtggaatattttagaactcggttgcctttgaggatgtggctgtgaacttcacccaggaggagtgggctttgttggatccttcccagaagaatctctatagagacgtgatgcaggaaaccttcaggaatctggcttccataggtaagaatcacaatatttcttcacttagtcaatcagaaagtaagtgtttcatggttttcaatgtcattccatgatttggaatgtacaaagggaacactttgatgagtgaatgaggcatggacccagtgtgcaatgactcttgtttcttagtctattttttaattgtgataacagaatacctgatcctgggtaatttacaaaaatagttttatattggtttatgattctagtggctagaaagtccaaggttgcatctggtaaggacctgatgctacttcagctcagggcatgaagcagaagcaagtgggcatgtgcaaagggatcacagcaagaaaataagagcaattcttgggagctaaacttgcttttataacaacctgccctctggcaactaattcagtcttgcaagagtaagagaaaacatgcattcccaagcaaggacattaatctattaataaggaatctgtacccataacacaaacaatgttgccatattgatgaccaaattacagcattcattttggtgggtacaaaccatatccccaacatagcacctagagtctagtaatttttctataatttcaaataatttgtaatatatttctgggtctacattttaggaaacaaaggggaagaccagagcattgaagatcagtacaaaaattcttcaagaaatctaaggtaatttgccctcacaagaggaagcagtcccccttggggtcaattgtagtatataatcatatgtttaaagcaaacaaggccaggcatggtggctcacacctgtaatcccagcactttgggatgctgaggtgggcggatcacctgaggtcaggagtttgagaccagcctgaccaacacggcaaaaacccgtctctactaaaaatacaaaattagcccagtgtgatggtgcatgcctgtaatccctgctgtttgggaggctgaggcaggagaattgcttgaacctgggaggcggaggttgcaatgagctgaggttgtgccattgcactccagccagggaaacaagagcaaaacaccatctcggggaaaaaaaaaaaccaagcaaacaaaataaattaagacaacatcaatttattctcaaagtttttaccaaaaatatatacttcaatgtaacatggatgttaaatgtttgcaaaatagttcacttgaaaacagtattgttttagtctgcttgcattgctataaaggaatacctaggctgagtaatttataaggaagagttttatttggctaatggttctgcaggctttatcagaagcataaagccagcatctgcttctgatgaggacctcagagagcttccagtcatggcagaaggggaaggggagctgacatgtcacatggggagagaaggagcaagagagagagaaaggaagtgccaaagtttttggggtttttttgttttgttttgttttgtttgagatggagtctctctctggtatccagactggagtgcagtggtgcagtcttggctcactgcaacctccgcctcccgggttcaagtgattctccggactcagtctcccaagtagctgggattacaggaacatgccaccacacccagctaatttttgtatttttagtagagacagcatttcaccatgttggccaggctggtcttgaattcctgacctcaagtgatctgcccaccttggcctcccaaagtgctgggattacaggcgtgagccaccatgcccgaccattttttttttttttttaacaatcggatctttcaggaactaatagagcgagaagtcactcattaccacaacagtgccacttatgtggaatctgctcccatgacccaagcacctcacaccaggtcatacctccaacatgaggatcaaatttcagcatgaaatttgaagggagaaaatacccaaactgtattcaatactaagaaactgcatatgagaatattactgtattgttaatagctataggggagggagccatgttgtagactaatcaatccatttatgttcaatttgtttatgttagaaaacctgcactttctctgatattggtagcagtgtaagttcagacttagtaataaaagaaaataactaataaaccattaatgatggggttgtcattttttgcagaagtcatatgatagacatactgtgtaaaattaaataagtcagtgtagaaaaacctccagatgccaaattttaatctgaacaaaaaaattcctgctagagtaaaaccacatgaatgcattgtgtgtgaaaaattcttcatacgtcattcatcccttcataggcacatcatatctcattctggaaacaacccatatgggtgtgaggaatgcggaaagaagccatgtacatgtaaacaatgtcagaaaacttccctttctgtcacaagggttcacagagacacagtaatgcacactggaaatggacattatggttgtacaatatgtgagaaagtttttaatattcccagttcatttcagatacatcagagaaatcacactggagagaaaccctatgaatgtatggaatgtgggaaagccttaggtttttcccgttctcttaatagacataaaaggattcacactggagaaaaacgctatgaatgtaagcaatgtgggaaagccttcagtcgttccagtcaccttcgtgaccatgaaagaactcatactggagagaaaccctatgaatgtaagcactgtgggaaagccttccgttactccaattgccttcattaccatgaaagaactcacactggagagaaaccttatgtgtgcatggaatgtggcaaagctttcagttgtctcagttccttgcaaggacatataaaggctcatgctggtgaagaaccctatccatgtaagcaatgtgggaaagccttcagatacgccagttcccttcagaaacacgagaaaactcatattgcacagaaaccctatgtatgtaacaattgtggtaaaggcttcagatgttccagttcccttcgtgaccatgaaaggactcatactggagagaaaccctatgaatgtcagaaatgtggcaaagcctttagtcgtgctagtaccctttggaagcataaaaaaactcatactggagaaaagccctataaatgtaaaaaaatgtaaaggctttaatcactacagtttttgtcaaaaacatgaacagtcacatacttgagagaaactgtgaatgtaaggtgtaggaaagtacttaattttcccagatttcctcaaatacatgaaacgaatcaaact

SEQ ID NO: 2 - DNA, 20 bp, Artificial Sequence
  Primer
tgaggatgtggctgtgaact

SEQ ID NO: 3 - DNA, 20 bp, Artificial Sequence
  Primer
actggaacgactgaaggctt

SEQ ID NO: 4 - DNA, 32 bp, Artificial Sequence
  Primer
ggcaggatcttcacctgttgaccaacatgcct

SEQ ID NO: 5 - DNA, 30 bp, Artificial Sequence
  Primer
ccaaccccttcctcctacatagttggcagt

SEQ ID NO: 6 - DNA, 24 bp, Artificial Sequence
  Primer
cgccttcttgacgagttcttctga

SEQ ID NO: 7 - DNA, 33 bp, Artificial Sequence
  Primer
ggtgcttgagtagtgttgaatctcagtggacca

SEQ ID NO: 8 - DNA, 21 bp, Artificial Sequence
  Primer
attccccttcaagatagctac

SEQ ID NO: 9 - DNA, 21 bp, Artificial Sequence
  Primer
aatgatcaactgtaattcccc

SEQ ID NO: 10 - DNA, 19 bp, Artificial Sequence
  Primer
gccgccgggatcactctcg

SEQ ID NO: 11 - DNA, 19 bp, Artificial Sequence
  Primer
ccagccgccgtcgcaactc

SEQ ID NO: 12 - DNA, 20 bp, Artificial Sequence
  Primer
tcggtctcatcttcattccc

SEQ ID NO: 13 - DNA, 20 bp, Artificial Sequence
  Primer
ggaaggctctgttccggtat